(19) United States
(12) Patent Application Publication
(10) Pub. No.: US 2020/0325239 A1
(43) Pub. Date: Oct. 15, 2020

(54) Modulation of Aminopeptidase N/CD13 and Rheumatoid Arthritis

(71) Applicant: THE REGENTS OF THE UNIVERSITY OF MICHIGAN, Ann Arbor, MI (US)

(72) Inventor: David A. Fox, Ann Arbor, MI (US)

(21) Appl. No.: 16/304,658

(22) PCT Filed: Jun. 9, 2017
(86) PCT No.: PCT/US17/36833

§ 371 Date: Nov. 26, 2018

Related U.S. Application Data
(60) Provisional application No. 62/348,739, filed on Jun. 10, 2016.

(51) Int. Cl. C07K 16/28; G01N 33/543; G01N 33/68; A61P 19/02

(52) U.S. Cl. CPC C07K 16/2896; G01N 33/543; A61K 2039/505; A61P 19/02; C07K 2317/76; G01N 33/68

(57) ABSTRACT

The disclosure provides materials and methods useful in modulating the course of autoimmune disorders that are monocyte-dependent and/or angiogenesis-dependent by administering inhibitors of aminopeptidase N/CD13.

### CROSS REFERENCE TO RELATED APPLICATION

This application claims priority to Provisional U.S. Patent Application No. 62/348,739, filed Jun. 10, 2016, which is hereby incorporated by reference in its entirety.

### STATEMENT OF GOVERNMENT SUPPORT

This invention was made with government support under NIH/NIAMS AR38477 awarded by the National Institutes of Health. The government has certain rights in the invention.

### BACKGROUND

Rheumatoid arthritis (RA) is a chronic inflammatory disorder in which infiltration of monocytes (MNs)/macrophages plays an essential role in its pathogenesis (2-4). Leukocyte ingress into the inflammatory sites is mediated by cytokines and growth factors, such as tumor necrosis factor-α (TNF-α), interleukin-1β (IL-1β), IL-6, interferon-γ (IFN-γ), IL-15, IL-23, transforming growth factor-β (TGF-β), monocyte chemoattractant protein-1 (MCP-1/CCL2), and IL-17 (5-11). Once MNs are recruited into the synovial membrane, they secrete proinflammatory and proangiogenic factors that result in proliferation of the synovial tissue (ST) and further MN migration. The secretion of proinflammatory and proangiogenic cytokines by MN/macrophages results in the proliferation and growth of the ST membrane which leads to persistence of the inflammatory response in RA. These studies provide evidence that MNs/macrophages play a key role in RA.

Angiogenesis contributes to pannus development and proliferation of the inflamed RA ST by providing nutrients and furthering the ingress of inflammatory cells (12-14). The role of angiogenic factors and cytokines, such as vascular endothelial growth factor (VEGF), basic fibroblast growth factor (bFGF), interleukin-8 (IL-8), TNF-α, IL-1β, MCP-1/CCL2, TGF-β, and IL-17 in RA is well established (15-24). Targeting VEGF, VEGFRs, and bFGF reduces arthritis onset, severity, and joint angiogenesis in mouse collagen-induced arthritis (CIA), adjuvant induced arthritis (AIA) in rats, and antigen-induced arthritis in rabbits (25-30). TNF-α and IL-1β exert indirect proangiogenic effects in RA by increasing the secretion of VEGF by ST fibroblasts (31, 32). These reports suggest that angiogenic factors are critical in the pathogenesis of RA.

Aminopeptidase N/CD13 (EC 3.4.11.2), a metalloproteinase of the M1 family, is a Zn²⁺-dependent ectoenzyme that cleaves the N-terminal peptide from its substrates (1-4). CD13 has been linked to the pathogenesis of a variety of immune-mediated conditions including rheumatoid arthritis (RA), scleroderma, psoriasis, and chronic graft-versus-host disease (2-8). In addition to RA, CD13 has also recently been implicated in osteoarthritis (OA) through a role on chondrocytes (9). CD13 is primarily a cell-surface molecule that was originally identified on myeloid cells (1), but is now known to be expressed by other cell types, including fibroblast-like synoviocytes (FLS; 10). It has also been identified in soluble fractions of biological fluids. CD13 is upregulated in RA synovial fluid compared to OA synovial fluid, normal human serum, or RA serum (10). CD13 is also found in FLS culture supernatants, demonstrating that CD13 is released from FLS (10). CD13 has been identified as a truncated soluble protein in human serum by Western blot; however, because CD13 is highly expressed on the cell surface, extracellular vesicles, which can reflect the protein composition of the cell surface, are another potential source of CD13 in cell free fractions (11, 12).

Extracellular vesicles include a variety of small vesicles such as exosomes, microparticles, and apoptotic bodies. Apoptotic vesicles are released by dying cells and microparticles are released primarily from platelets, but exosomes can be released from a wide variety of cell types, including FLS (13). Exosomes are small (40-120 nm diameter) lipid bilayer vesicles that typically express a surface profile similar to that of the cells from which they are released (13). CD13 has been previously demonstrated on exosomes from microglial cells and mast cells (14, 15).

Rheumatoid arthritis, including refractory RA, is a crippling disease associated with rapid joint destruction. Therefore, there is a significant need to explore the role of other cytokines and factors, such as CD13, in treating RA patients. Further, a need continues to exist in the art for materials and methods to modulate the expression or activity of biomolecules involved in autoimmune disease, such as rheumatoid arthritis.

### SUMMARY

The disclosure provides materials and methods for modulating the course of autoimmune diseases that are monocyte-dependent and/or angiogenesis dependent. Targeting CD13 is expected to provide a superior approach to treating angiogenesis- and/or monocyte-dependent chronic inflammatory diseases such as RA because of the ability of CD13 to induce: a) chemotaxis of cytokine activated T cells (Tcks), monocytes (MNs) and endothelial cells (ECs) in vitro; b) fibroblast-like synoviocyte (FLS) cell proliferation and migration; c) Tck chemotaxis through a G protein-coupled receptor (GPCR) (1); d) EC tube formation on Matrigel; and e) angiogenesis in a mouse Matrigel plug assay. MN ingress and angiogenesis are two key factors involved in the pathogenesis of RA. Disclosed herein is experimental evidence demonstrating the direct involvement of recombinant CD13 in RA angiogenesis and MN ingress as well as in an animal model of RA.

In one aspect, the disclosure provides a method of generating a binding partner specifically recognizing a cell-surface protein of a synovial fibroblast comprising: (a) contacting at least one synovial fibroblast with Interleukin 17 to stimulate the at least one synovial fibroblast; (b) administering an immunogenic amount of the at least one synovial fibroblast to an immunocompetent host organism; and (c) obtaining an antibody specifically recognizing a cell-surface protein of the synovial fibroblast. In some embodiments, the binding partner is a monoclonal antibody or binding fragment thereof, such as antibody 1D7, or a binding fragment thereof. In some embodiments, the cell-surface protein is localized on an episome, such as an episome that is 30-130 nm in diameter. In some embodiments, the cell-surface protein is a human protein. In some embodiments, the cell-surface protein is CD13.

Another aspect of the disclosure is drawn to a method of measuring the concentration of CD13 in a sample comprising (a) contacting the sample with an anti-CD13 antibody, or binding fragment thereof, produced by a method disclosed herein; and (b) measuring the concentration of CD13 in the sample based on the extent of binding of anti-CD13 antibody, or binding fragment thereof. In some embodiments, the concentration is measured using an ELISA assay.

In another aspect, the disclosure provides a method of treating an autoimmune disorder comprising administering an effective amount of an inhibitor of CD13. In some embodiments, the inhibitor is an anti-CD13 antibody or binding fragment thereof. In some embodiments, the anti-CD13 antibody is antibody 1D7 or a binding fragment thereof.

Another aspect of the disclosure is directed to a method of treating an autoimmune disorder in a subject comprising administering an effective amount of an inhibitor of CD13 cleavage from a cell membrane. In some embodiments, the autoimmune disorder is rheumatoid arthritis. In some embodiments, the cell membrane is an exosome membrane. In some embodiments, the inhibitor reduces the protein cleavage activity of a matrix metalloproteinase. In some embodiments, the matrix metalloproteinase is selected from the group consisting of MMP14, MMP15, MMP16, MMP17, ADAM10, ADAM15 and ADAM17, such as MMP14. In some embodiments, the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.

Another aspect of the disclosure provides a method of inhibiting the migration of a cytokine-activated cell in a subject comprising administering an effective amount of a CD13 inhibitor. In some embodiments, the cell is an endothelial cell, a monocyte or a T-cell. In some embodiments, the inhibitor is an anti-CD13 antibody or binding fragment thereof, such as antibody 1D7 or a binding fragment thereof. In some embodiments, the CD13 inhibitor is an inhibitor of a matrix metalloproteinase, such as MMP-14. In some embodiments, the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.

Still another aspect of the disclosure is drawn to a method of inhibiting angiogenesis in a subject comprising administering an effective amount of a CD13 inhibitor. In some embodiments, the inhibitor is an anti-CD13 antibody or binding fragment thereof, such as antibody 1D7 or a binding fragment thereof. In some embodiments, the CD13 inhibitor is an inhibitor of a matrix metalloproteinase, such as MMP-14. In some embodiments, the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.

Other features and advantages of the disclosure will be better understood by reference to the following detailed description, including the drawing and the examples.

### BRIEF DESCRIPTION OF THE DRAWING

FIG. 1. CD13 is found as a soluble protein and on extracellular vesicles. (A) Supernatants from 3 flasks of RA fibroblasts were concentrated through a 30K centrifugal filter. RA synovial fluid was diluted with PBS (4:1), and 10 ml of plasma was obtained from a healthy individual. Vesicle fractions were isolated from the samples by serial centrifugation. Supernatants from the final centrifugation were collected as the soluble protein fraction. The vesicle pellet was resuspended in 1 ml PBS. CD13 was measured by ELISA and aminopeptidase activity was analyzed through cleavage of L-leu-AMC. Data were normalized to concentration in original fluid. mean±SEM n≥3. (B) Exosomes were lysed and purity was confirmed by Western blot for flotillin-1 and CD9. Single bands appeared at expected sizes with a weak band for CD9 and a strong band for flotillin-1, confirming exosomes from FLS. (C) A discontinuous optiprep gradient was created in seven fractions from 1.268 g/ml to 1.031 g/ml. 500 μl of the resuspended vesicles was layered onto the top of the gradient. The loaded gradients were centrifuged at 100,000×g for one hour. Fractions were collected in reverse. Fractions were washed in PBS at 110,000×g for two hours and the pellets were resuspended in 500 μl PBS. Soluble CD13 is present in the first supernatant separation, exosomes are in fractions 3-5, and other extracellular vesicles are shown from the other fractions. CD13 was observed in all three fractions. Data were converted to percentage of total fluid CD13. % total n≥3.

FIG. 2. Metalloproteinases cleave CD13 from the surface of FLS. Five different protease inhibitors were added to FLS cultures covering all classes of proteases. (A) The only inhibitor to decrease shedding of CD13 into the supernatant of the cultures was GM6001. (B) No significant decreases were seen in cell lysate CD13 concentrations. Cultures were incubated with serum-free media containing protease inhibitors for 48 hours: Pepstatin A (aspartic) 10 μM (labeled “Pepestatin A (Aspartic) in the figure), Aprotinin (serine) 100 nM, Leupeptin (serine/cysteine) 10 μM, GM6001 (metalloproteinase) 25 μM, and E-64 (cysteine) 10 μM. (C) TIMP-2 (0.6m/ml) inhibited the secretion of CD13 from RA FLS while TIMP-1(0.6m/ml) did not. FLS from 3 different RA patients were used. Secretion was measured by ELISA and optical density (OD) of the samples was measured. (n=3) mean of % change±SEM*p≤0.05 **p≤0.01***p≤0.0001.

FIG. 3. MMP14 knockdown partially inhibits the shedding of CD13 from FLS. (A) MMP14 mRNA was almost completely removed by transfection with MMP14 siRNA. ADAM10, ADAM15, and ADAM17 knockdown constructs each also almost completely removed their respective mRNAs with appropriate siRNA transfection. None of the siRNAs (MMP14, ADAM10, ADAM15, or ADAM17) had an off-target effect on MMP1. n=3 (B) Shedding of CD13 into FLS culture supernatant was inhibited by knockdown of MMP14. Further inhibition was seen with GM6001. CD13 in FLS lysate was not changed by knockdown of MMP14, and significantly increased in GM6001 treated FLS. FLS were grown to confluence, then transfected with siRNA using an Amaxa nucleofector kit. Cells were grown to 75% confluence, then switched to serum-free growth media (Peprogrow) supplemented with 1 ng/ml IL-1 and 10 ng/ml TNFα for 48 hours with or without 25 μM GM6001. (C) Cells were incubated for 1 hour at room temperature with anti-CD13-FITC (1D7) 1 μg/100 μl and anti-MMP14-PE (128527) at 1.67 μg/100 μl. Figures shown were taken at 1000×. The four panels show: top left, MMP14 alone (red); top right, CD13 alone (green); bottom left, co-localization (co-localization in yellow); and bottom right, co-localization analysis by ImageJ (white=overlapping red and green pixels). All images were background corrected using DAPI alone and MsIg-FITC and MsIg-PE for threshold limits. Representative of n=6, *p≤0.05 **p≤0.005 ***p≤0.0001 mean±SEM (n≥2).

FIG. 4. CD13 is upregulated in FLS at the mRNA level but the effects on CD13 protein levels are varied. FLS were stimulated over a time course of 0-72 hours with IFNγ (1000 U/ml), TNFα (10 ng/ml), or IL-17 (10 ng/ml). Cells were harvested and processed for either mRNA (A), surface expression (B), total cell lysate CD13 (C), or total CD13 in supernatant (D). mRNA was measured by qRT-PCR. CD13 was measured on the surface with anti-CD13 (1D7) and flow cytometry. Cell lysate and supernatant CD13 concentrations were measured by CD13 ELISA. Gating was done to isolate the major cell population and exclude debris and dead cells. Data is expressed as a ratio to unstimulated FLS at the same time point in either ΔΔCt normalized to GAPD (mRNA), mean fluorescent intensity corrected for background florescence with MsIg staining (surface), or CD13 concentration in lysate or supernatant. A n=3 B-D n=1 *p≤0.05.

FIG. 5. CD13 chemical inhibitors or antibodies significantly slow growth and migration of RA FLS in vitro. FLS were seeded on 96-well plates overnight. An Essen Incucyte system was used for both growth (A) and migration (B) assays. Growth was calculated as the difference in percent confluence from time 0. Migration was measured in a scratch-wound assay using relative wound density. All data are expressed as a ratio to untreated FLS of the same cell line. mean±SEM n≥4 (*p≤0.05).

FIG. 6. (A) An example of NanoSight analysis. The left panel shows a single run of synovial fluid extracellular vesicles with concentration on the y-axis and size of particle on the x-axis. The background shows a frame of the count. Red crosses are a counted particle. The right panel shows an average of all runs for a synovial fluid with concentration on the y-axis and size of particle on the x-axis. The dashed lines show approximate exosome size. (B) A discontinuous Optiprep gradient was created in seven fractions from 1.268 g/ml to 1.031 g/ml. 500 μl of the resuspended vesicles was layered onto the top of the gradient. The loaded gradients were centrifuged at 100,000×g for one hour. Fractions were collected in reverse. Fractions were washed in PBS at 110,000×g for two hours and the pellets were resuspended in 500 μl PBS.

FIG. 7. Fluorescent staining of FLS shows co-localization of CD13 and MMP14. RA FLS were grown to 90% confluence on 8-well glass chamber slides. Cells were fixed with 1% Formalin and blocked with Fc block (10% human serum/10% mouse serum in PBS). Cells were incubated for 1 hour at room temperature with (A) anti-CD13-FITC (1D7) 1 μg/100 μl or (G) anti-CD90-FITC 1 μg/100 μl and (B and H) anti-MMP14-PE (128527) at 1.67 μg/100 μl (appropriate isotype controls and single staining were also done). The nuclei were counter stained with (C and I) DAPI at 1 μg/ml. Overlapping signals are shown in D and J. Cells were mounted using anti-fade media. Confocal microscopy was performed using an Olympus microscope. All images corrected for background—thresholds determined by DAPI alone, MsIg-FITC alone, and MsIg-PE alone. Co-localization analysis was run using an ImageJ add-in, red and green pixels that co-localize are shown in white (E, CD13-MMP14; K, CD13-CD90) and the scatter plots of co-localization are shown in F and L respectively. Representative of n=6.

FIG. 8. Examples of scratch-wound images show decrease in FLS migration with actinonin and anti-CD13 (1D7) compared to irrelevant isotype control (anti-CD3). FLS were seeded on 96-well plates overnight. An Essen Incucyte system was used for scratch wounds and migration measurements. Migration was measured in a scratch-wound assay using relative wound density. Representative of n≥4.

FIG. 9. CD13 protein levels were measured by performing ELISAs with RA synovial fluids (SFs) and osteoarthritis (OA) SFs. The data reveal that RA SFs have significantly higher concentrations of CD13 compared to OA SFs.

FIG. 10. Human dermal microvascular endothelial cell (HMVEC) chemotaxis was performed in a modified Boyden chamber using CD13 at various concentrations. CD13 was found to induce HMVEC migration in a dose-dependent manner. CD13 induced HMVEC migration from 500-2000 ng/ml (p<0.05). Data represent the mean of 3 individual experiments±SEM. Three high-power fields (hpf) (×400) were counted in each replicate well and results were expressed as cells per hpf. PBS served as negative control.

FIG. 11. Human dermal microvascular endothelial cell (HMVEC) tube formation was assessed on growth factor-reduced Matrigel (GFR, BD Biosciences) with CD13. EC tubes formed in response to CD13 were almost two-fold more numerous compared to PBS, a negative control. Arrows indicate the number of tubes formed in each group. n=number of replicates in each group. The number of tubes formed was quantitated by an observer blinded to the experimental groups.

FIG. 12. A 3D EC spheroid assay was performed using human dermal microvascular endothelial cells (HMVEC) and RA synovial tissue (ST) fibroblasts. Panel A shows almost no sprouts formed in response to negative control, while panel B shows EC sprouts formed in response to basic fibroblast growth factor (bFGF).

FIG. 13. To test the effect of CD13 in angiogenesis in vivo, Matrigel plug assays were performed using C57Bl/6 mice. Each mouse was given a subcutaneous injection of sterile GFR Matrigel (500 μl/injection) containing either CD13 or PBS, a negative control. Matrigel plugs were harvested after 7 days. Hemoglobin (Hb) was determined. There was more than a four-fold increase in Hb, an indirect correlate of neovascularization, in the plugs containing CD13. Results are represented as the mean+SEM and *p<0.05 was considered significant. n=number of mice per group.

FIG. 14. To evaluate the contribution of CD13 to RA inflammation by recruiting MNs, normal human MN chemotaxis assays were performed in modified Boyden chambers. CD13 was found to induce a concentration-dependent increase in MN migration. PBS served as negative control. n=number of experiments. The results are expressed as mean+SEM and *p<0.05 was considered significant. n=number of replicates.

FIG. 15. RA STs were cryosectioned and immunofluorescence was performed to detect the expression of CD13 on FLS and synovial blood vessels. Primary antibodies were anti-human CD13 (1D7, 10 μg/mL), rabbit anti-human Cadherin 11 (Zymed 10 μg/mL), a FLS marker, rabbit anti-human von Willebrand factor, an endothelial cell marker, and DAPI (nuclear stain). This staining showed that CD13 is highly expressed on synovial blood vessels and fibroblasts. Expression of CD13 on synovial monocytes has also been observed.

FIG. 16. To determine the blocking effect of CD13 antibody in a functional assay, monocyte (MN) chemotaxis was assessed in modified Boyden chambers. MNs were isolated from wild-type C57Bl/6 mouse spleens. Five mouse spleens were used to harvest MNs. Miltenyi beads were used to isolate splenic MNs. Recombinant CD13 was incubated with rat anti-CD13 (5 μg/ml) for 30 minutes before performing MN chemotaxis assays. It was found that rat anti-mouse antibody from Abcam-blocked CD13 mediated mouse MN migration but ant-CD13 antibody from GenTex did not inhibit CD13 mediated MN migration. Results represent the mean+SEM and *p<0.05 was considered significant. n=number of replicates.

FIG. 17. Chick chorioallantoic membrane (CAM) assay. In this assay, articular cartilage fragments from rabbits were cultured with RA FLS and then implanted on top of the CAM of chick embryos (64). Invasion of the FLS into the cartilage with increased angiogenesis was observed by immunohistochemistry. We will examine the role of CD13 in the angiogenesis that accompanies FLS cartilage invasion by using FLS transfected with CD13 siRNA or control siRNA in the assay.

### DETAILED DESCRIPTION

The experiments disclosed herein reveal the expression and function of CD13 on human RA FLS. The effects of three pro-inflammatory cytokines linked to RA on CD13 expression in RA FLS were examined. Additional data establishes how CD13 is released from FLS. The possibility that CD13 is present on exosomes or other extracellular vesicles derived from FLS and other human cell types was also explored, and soluble versus vesicle-bound CD13 were measured in sera, synovial fluids, and FLS culture supernatants. In addition, the autocrine effects of CD13 on RA FLS were examined.

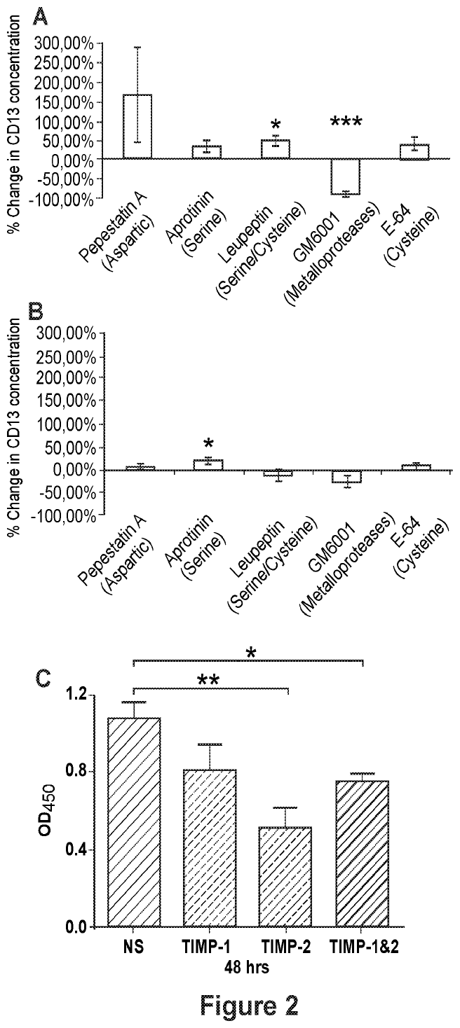

The work described in the following examples was undertaken with the conviction that CD13 plays an important role in RA as a T cell chemoattractant (10), and the disclosed data indicate additional roles for CD13 in the pathogenesis of RA. CD13 has been found in the cell-free portions of various biological fluids, including FLS culture supernatant and synovial fluid (6,10,29,30). There are three possible mechanisms by which FLS may release CD13: secretion through exocytosis of sCD13, protease-mediated cleavage from the cell surface, and secretion of CD13 on the surface of extracellular vesicles such as exosomes. Differential ultracentrifugation was used to distinguish between vesicle-associated and soluble CD13, and it was found that CD13 was present both on exosomes and as a soluble molecule. As a strongly expressed cell-surface structure, we expected CD13 cleavage to be more likely than secretion of sCD13. This expectation was realized by the observation that sCD13 in serum is truncated and lacks the intracellular and transmembrane domains, indicating cleavage from the cell membrane (12). This mechanism was examined using inhibitors specific for different classes of proteases: pepstatin A (aspartic acid), aprotinin (serine), leupeptin (serine/cysteine), GM6001 (metalloproteinases), and E64 (cysteine). The data indicate that CD13 is cleaved from FLS by metalloproteinases (FIG. 2A). While there are several sub-classes, there are two main groups of metalloproteinases, i.e., (1) matrix metalloproteinases (MMPs), and (2) a disintegrin and metalloproteinase (ADAMs). Transmembrane proteases, which are known to participate in cleavage and release of proteins anchored in the membrane, are most likely responsible for release of CD13. Several of these proteases are members of the metalloproteinase family (MMPs14,15,16,17 and many ADAMs). Furthermore, the experiments with TIMPs described below indicate that membrane-type matrix metalloproteinases (MT-MMPs) are more likely to be mediating the shedding of CD13 than are soluble MMPs. Thus TIMP-1, which poorly inhibits MT-MMPs but inhibits soluble MMPs effectively, did not significantly suppress cleavage of CD13, while TIMP-2, which inhibits both MT-MMPs and soluble MMPs, did significantly decrease cleavage of CD13 (FIG. 2C) (31).

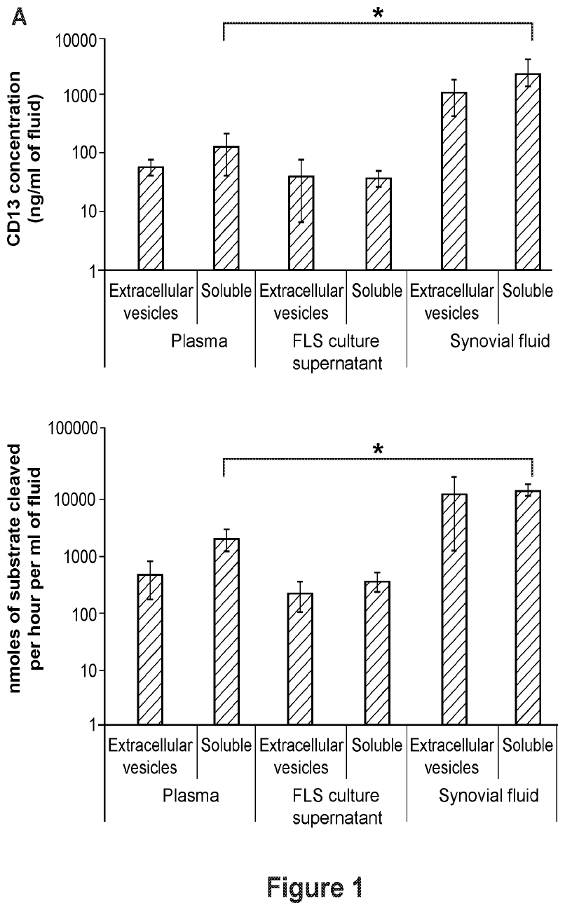

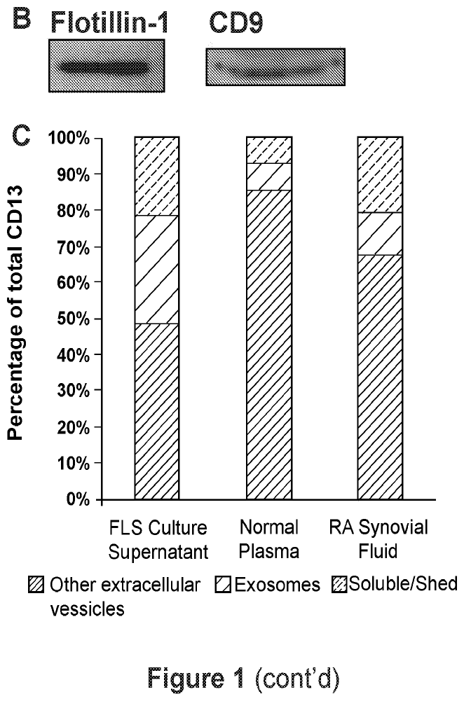

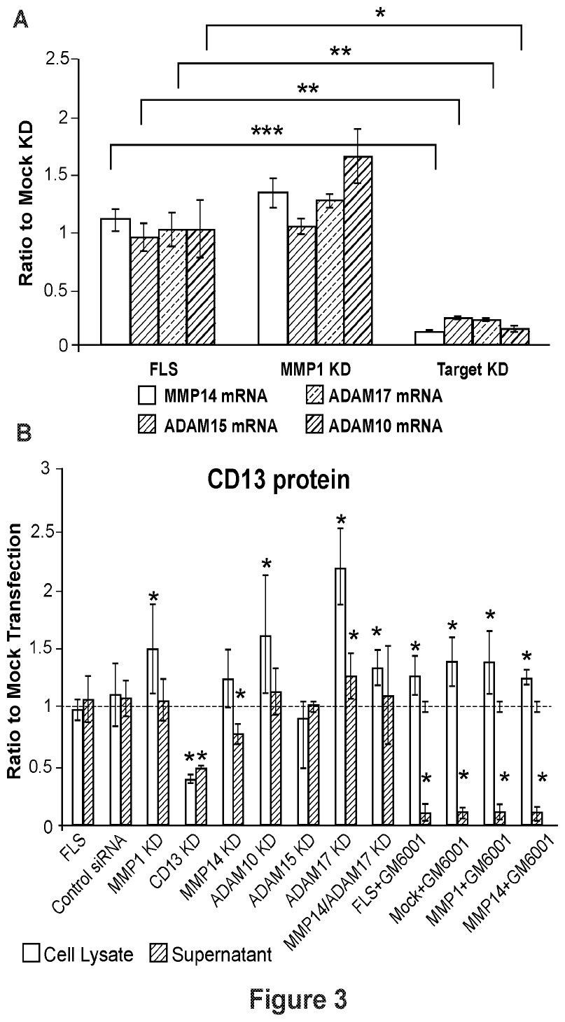

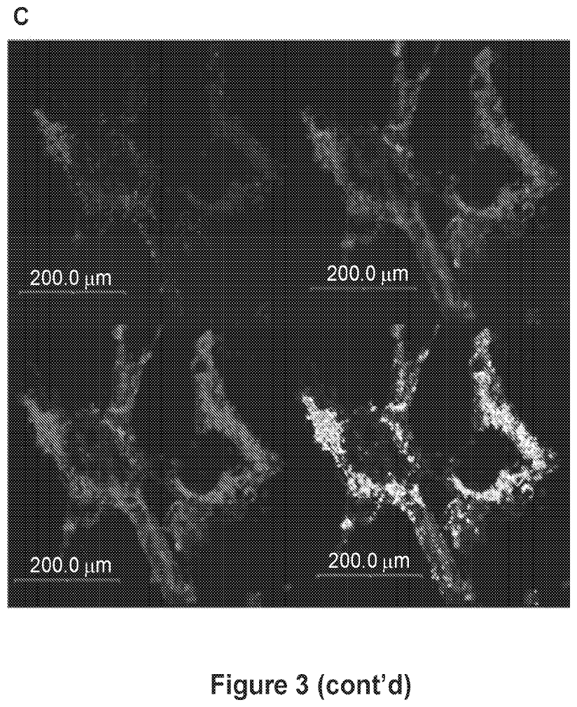

Of the MT-MMPs, MMP14 is found at the highest amount on the surface of RA FLS (32,33). In RA, MMP14 has been linked to matrix degradation by FLS and osteoclast-mediated bone resorption (33). Multiple studies have shown that of the MMPs expressed by synoviocytes, MMP14 in particular is important as a type I and type II collagenase and is essential for invasion of cartilage by FLS(26-28,34). Moreover, as disclosed hereinbelow, siRNA inhibition of MMP14 resulted in a significant decrease in CD13 cleaved from FLS (FIG. 3C). While MMP14 knock-down (KD) results in only an approximately 23% decrease in CD13 in the supernatant, MMP14 is expected to be primarily involved in the release of soluble CD13. Soluble CD13 accounted for around half of the CD13 in FLS culture supernatants, with the other half from EVs (FIG. 1C). Thus, knockdown of MMP14 might be expected to only partially affect release of CD13 from FLS, if MMP14 controlled cleavage of CD13 from the FLS membrane, but not release in EVs. In addition, inhibition of all metalloproteinases by GM6001 further reduced the CD13 released into the supernatant. The data establish roles for various MMPs in release of CD13 on EVs, and a primary role for MMP14 in cleavage of membrane CD13 from the cell surface.

Many members of the metalloproteinase family (and especially ADAMs) have the same or similar substrates, and multiple metalloproteinases can be involved in the same biological functions (35-40). Even though collagenolytic activity is the best characterized example of a shared substrate, with most MMPs demonstrating this function, the similarity of cleavage sites and activity may carry over to other substrates (25). It is possible that other membrane-bound MMPs (e.g., MMP15, 16, or 17) are also involved in CD13 shedding. MMP15/MT2-MMP mRNA has been found in RA synoviocytes, and MMP16/MT3-MMP has been found on synovial tissue biopsies (26,33). Very little mRNA of either MMP15 or MMP16, however, was found in our RA FLS lines. The other possible group of CD13 sheddases is the ADAMs. This is supported by the observation that some soluble CD13 remains even after inhibition with TIMP-2 (FIG. 2C), given that TIMP-3 is the primary suppressor of ADAMs (31). The fact that TIMP-1 and TIMP-2 in combination did not completely inhibit shedding of CD13 indicated that metalloproteinases other than MMPs were involved, specifically ADAM family members. In particular ADAM17, ADAM15, and ADAM10 have been linked to the shedding of various proteins (36,40,41). ADAM17 has also been suggested to interact with CD13 on the surface of myeloid leukemia cells (19). ADAM15 has been found to be up-regulated in RA synovium compared to OA, is constitutively expressed by RA FLS, and has been linked to angiogenesis and FLS migration(42-44). Single-knockdown of ADAMs 10, 15, or 17, however, did not result in a decrease of CD13 in the culture supernatant (FIG. 3B). Based on the disclosures herein, it is apparent that metalloproteinases cleave CD13 from the FLS membrane, with MMP14 being the primary sheddase, while multiple other metalloproteinases likely act together to also shed CD13. In addition, metalloproteinases may act in an as yet poorly characterized role in the release of CD13⁺ EVs from FLS. This notion is supported by a report that GM6001 inhibited the release of exosomes from endothelial cells (45).

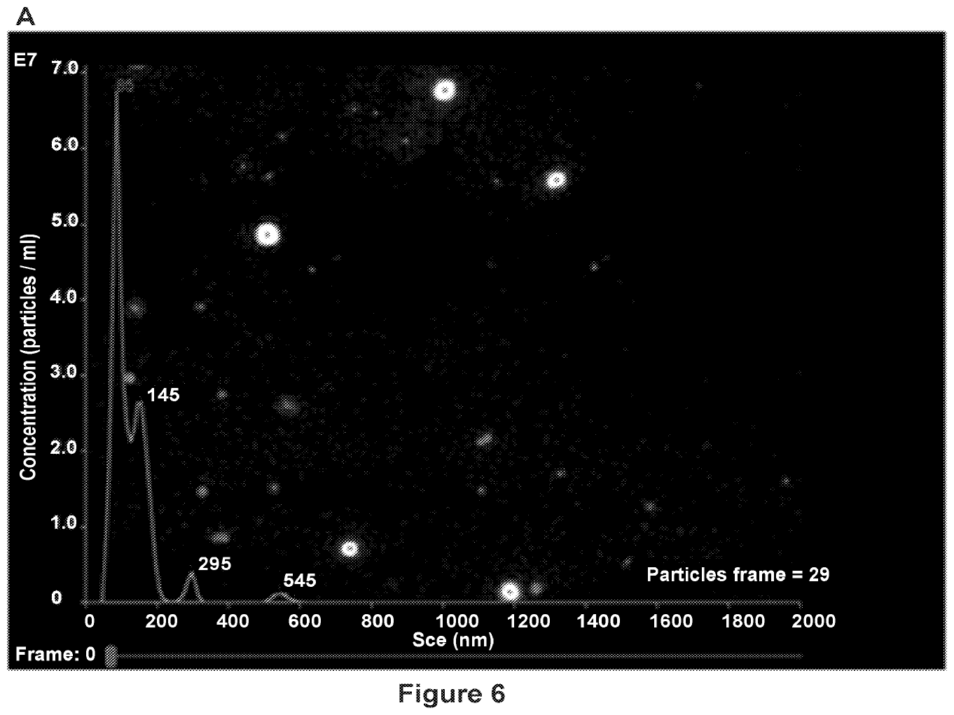

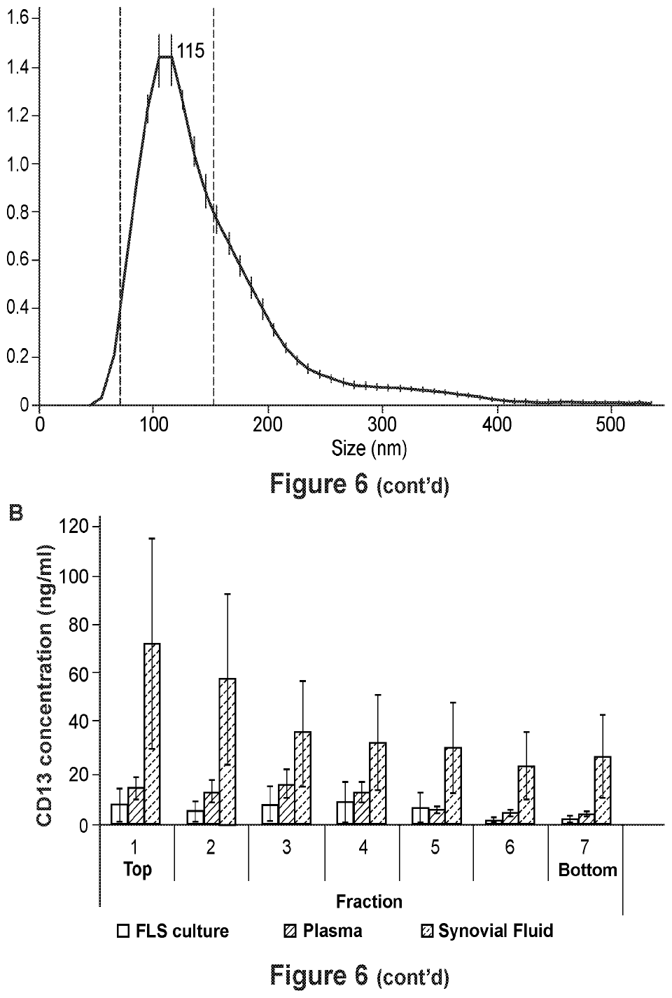

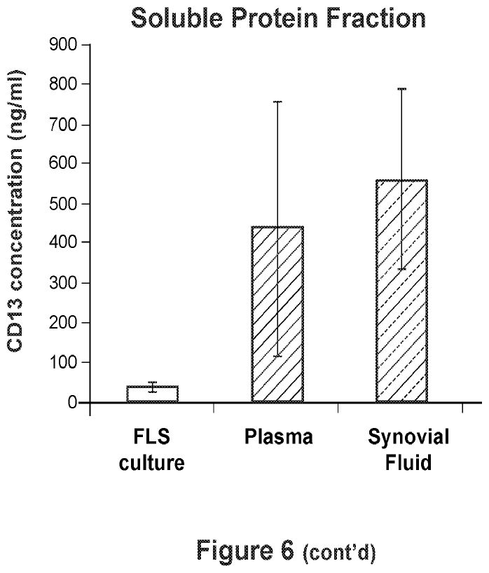

To confirm MMP14 as a cleaver of CD13, possible co-localization of CD13 and MMP14 on FLS was examined. CD13 and MMP14 have previously been found in similar cell surface domains, but their proximity has not been determined. Both CD13 (FLS) and MMP14 (breast carcinoma and glioma cells) have been found in caveolae-enriched lipid rafts (46,47). On the surface of FLS, CD13 and MMP were shown herein to co-localize and in some cells, a punctate pattern was observed, which is expected to be indicative of inclusion into lipid raft structures (FIG. 3C, FIG. 6). Overall, the data disclosed herein indicate that CD13 and MMP14 localize in similar areas on the FLS cell surface, consistent with MMP14 having a primary role in the cleavage of CD13.

CD13 was identified in vesicle fractions in plasma, synovial fluid, and FLS culture supernatant and as a soluble molecule (FIG. 1A). Nanosight counting revealed a predominance of exosome-sized vesicles in the CD13⁺ vesicle fractions. Differential ultracentrifugation revealed that CD13 was associated with vesicles at a density similar to that of exosomes. Further density separation identified CD13 at densities from 1.268 g/ml to 1.031 g/ml, indicating its presence on exosomes as well as other extracellular vesicles of similar density (FIG. 1B). One problem with the differential centrifugation method is that it can isolate other EVs or large protein aggregates of similar density to exosomes. The additional separation by density gradient, however, can distinguish between exosomes, other EVs, and protein aggregates. Apoptotic blebs float above a density of about 1.23 g/ml while exosomes float at 1.10-1.21 g/ml (48). The results herein demonstrate that CD13 is present as both a soluble molecule and as a membrane-bound molecule on extracellular vesicles derived from FLS.

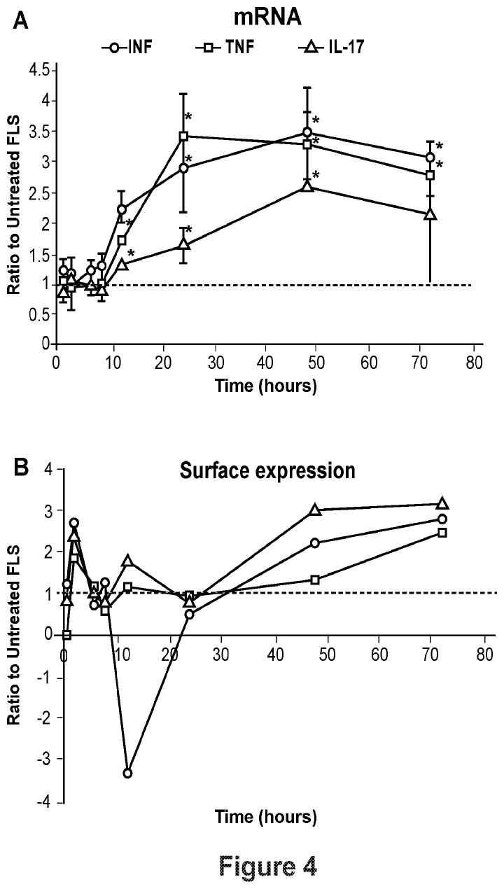

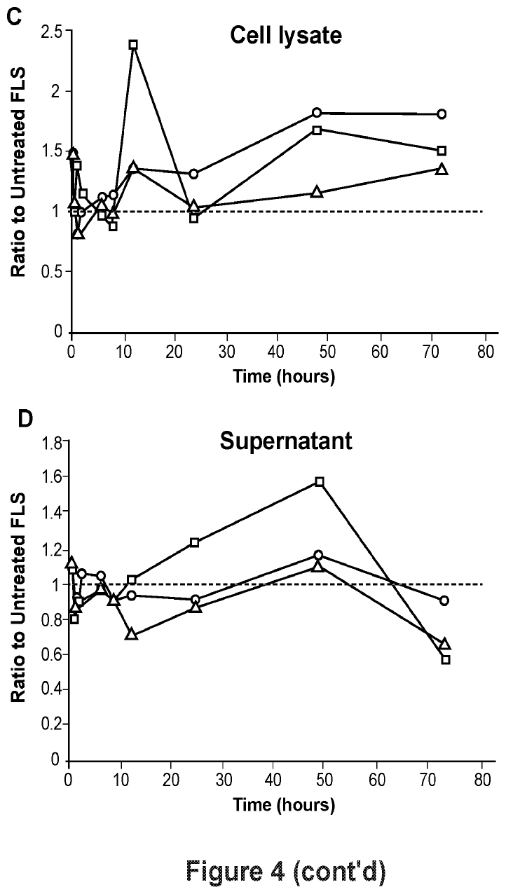

CD13 represents a significant portion of the T cell chemotactic ability of RA synovial fluid (10). Once in the joint, T cells are known to activate RA FLS through cell-cell interactions and the release of pro-inflammatory cytokines (49-51). This activation can result in greater production of chemokines by the FLS, resulting in a self-perpetuating, pro-inflammatory cycle (51). While there are no differences in CD13 expression between OA and RA FLS in culture, there is significantly more CD13 in RA than in OA synovial fluid (10). Without wishing to be bound by theory, one possibility is that pro-inflammatory cytokines produced by invading cells (T cells/monocytes) up-regulate CD13 in the RA synovium, but that under culture conditions, this up-regulation reverts to a baseline level. To determine whether CD13 expression could be a part of this inflammatory loop, the effect of three pro-inflammatory cytokines on CD13 expression by FLS was examined in the experiments described below. CD13 mRNA was upregulated by IFNγ, TNFα, and IL-17 in FLS. The intensity of CD13 protein expression on the FLS cell surface, however, did not match this regulation pattern. Even before the mRNA was upregulated (peak around 48 hours), the cytokines induced fluctuations in cell surface, total cell lysate, and supernatant CD13 (FIG. 4). Overall, it is apparent that IFNγ, TNFα, and IL-17 up-regulate CD13 mRNA, and also change both protein expression and localization, with distinct kinetics in individual RA FLS lines. A high degree of variability was seen within and between cell lines, indicating that this is a complex and dynamic process. CD13 can mediate phagocytosis in monocytic lineage cells, thereby also undergoing internalization (52,53). In mice, it has been shown that CD13 negatively regulates inflammation through co-internalization of TLR4 and CD13, thereby negatively regulating TLR4 signaling (54). While this may or may not be relevant in human FLS, it suggests that CD13 localization (cell surface versus other location) can be important in the control of inflammation. Thus, CD13 may be crucial to the fine balance of inflammation through both positive and negative regulation of inflammatory signals, depending on its localization. For example, on the cell surface, it may act to down-regulate TLR4 signaling while as a soluble molecule, it acts as a T cell chemoattractant (10,54). CD13 has been shown to be present in caveolae lipid rafts in both FLS and monocytes, which may indicate a mechanism for CD13 internalization that may contribute to inflammatory regulation in addition to the shedding demonstrated herein (47,55).

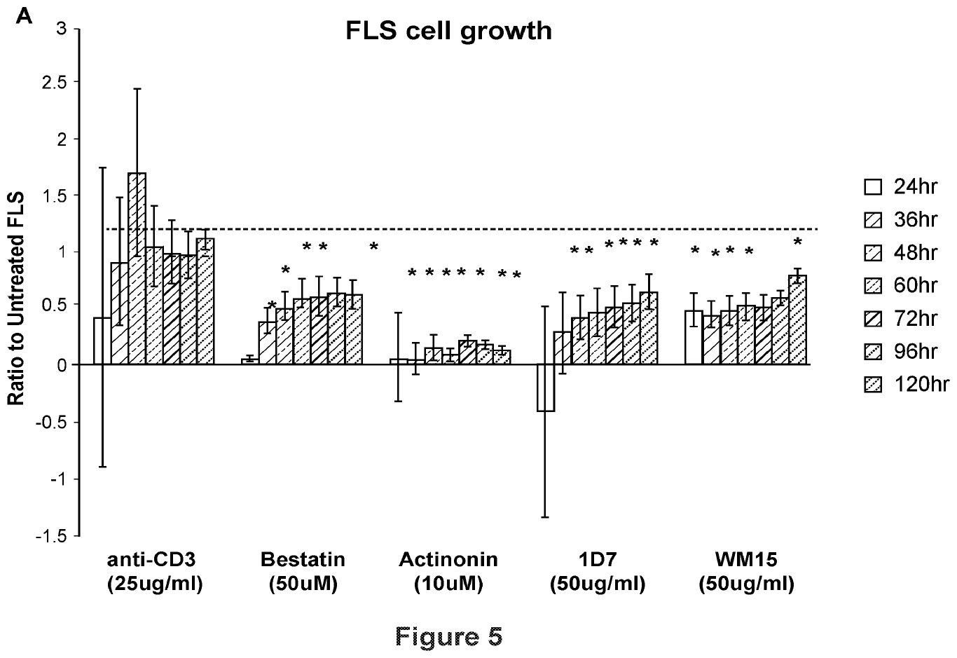

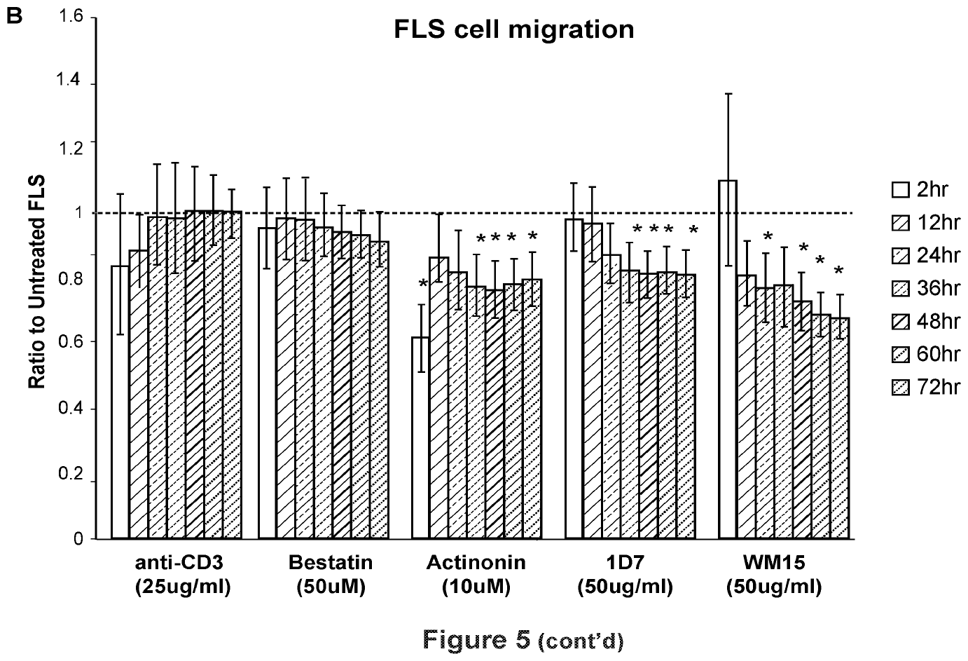

Another component of disease pathology in RA is aggressive outgrowth and migration of FLS, manifested clinically as synovial hyperplasia. Previous data has implicated CD13 in the migration but not proliferation of dermal fibroblasts (56). Data disclosed herein indicate a role for CD13 in the growth and migration of RA FLS (FIG. 5). However, it is uncertain whether this is dependent on soluble CD13 or cell-surface CD13. While inhibitors of CD13 enzymatic activity and anti-CD13 antibodies each inhibited FLS proliferation and migration, additional rhCD13 did not consistently affect the RA FLS. This may reflect the large amount of CD13 produced by the FLS that can act in an autocrine fashion. Because chemical inhibitors of CD13 enzymatic activity, i.e., bestatin and actinonin, inhibit FLS proliferation and actinonin inhibits migration, it appears that CD13 enzymatic activity may be necessary for these functions. Also, the specific antibody WM15, which inhibits CD13 enzymatic activity, inhibited proliferation and migration. However, an anti-CD13 antibody that does not inhibit enzymatic activity, i.e., 1D7, also inhibited FLS growth and migration (10). Without wishing to be bound by theory, the most likely explanation is steric hindrance. 1D7 does not block cleavage of the small molecule L-leu-AMC in the CD13 aminopeptidase activity assay; however, it may block the ability of CD13 to associate with larger substrates on FLS, thereby indirectly but effectively blocking enzymatic activity and cell growth or migration.

Bestatin was not found to inhibit FLS migration, although it did inhibit FLS growth. This may be due to the fact that bestatin is not specific for CD13. Bestatin may exert effects on other peptidases that counteract the effect on CD13. The functional role of CD13, however, is confirmed by specific anti-CD13 antibodies. These results demonstrate another potential pathogenic role for CD13 in RA through its effects on RA FLS, in addition to its function as a T cell chemoattractant in RA.

Overall, the data point to roles for CD13 in the pro-inflammatory milieu of the RA synovium. CD13 is upregulated by pro-inflammatory cytokines, and is released from FLS. Targeting of the molecules responsible for the release of CD13 (such as MMP14) is expected to be a point of regulation for inflammatory diseases such as RA.

The following examples illustrate embodiments of the disclosure.

### EXAMPLES

### Example 1

Materials and Methods

Cell Culture

All procedures involving specimens obtained from human subjects were performed under a protocol approved by the University of Michigan Institutional Review Board. Fibroblast-like synoviocytes (FLS) were cultured from human synovial tissue obtained at arthroplasty or synovectomy from RA joints by digestion with 1% collagenase and separation through a 7004 cell strainer (16). FLS were uniformly positive for the FLS marker Cadherin-11. The diagnosis of RA required at least four of the seven 1987 American College of Rheumatology criteria (17). FLS were maintained in Connaught Medical Research Laboratory (CARL) medium (20% fetal bovine serum (FBS), 2 mM L-glutamine, and 1% penicillin/streptomycin), and were used between passages 4 and 10. To avoid the confounding effect of serum CD13, cultures were moved to serum-free Dulbecco's Modified Eagle's medium/F-12 with Peprogrow serum replacement (Peprotech, Rocky Hill, N.J.) before harvesting. Some cultures were treated with protease inhibitors for 48 hours in serum-free medium: pepstatin A (Sigma-Aldrich, St. Louis, Mo.), aprotinin (Sigma-Aldrich, St. Louis, Mo.), leupeptin (Sigma-Aldrich, St. Louis, Mo.), GM6001 InSolution (EMD Millipore, Darmstadt, Germany), or E-64 (Thermo Scientific, Waltham, Mass.). Other cultures were treated with cytokines: recombinant human interferon-γ (rhIFNγ, 1U/ml), recombinant human tumor necrosis factor-α (rhTNFα, 10 ng/ml), or recombinant human interleukin-17 (rhIL-17,10 ng/ml) (Peprotech, Rocky Hill, N.J.) for 0, 0.5, 1, 2, 6, 8, 12, 24, 48, or 72 hours in serum-free medium.

Sample Preparation

Synovial fluid samples were treated with 0.05% Hyaluronidase (bovine testis, Sigma-Aldrich, St. Louis, Mo.) at one drop per 1 mL fluid for 5 minutes. Cells were lysed in cell lysis buffer (10% NP-40, 10% PMSF, 1% Iodoacetamide, and 0.1% E-64 in TSA) for one hour on ice and spun to remove debris. FLS culture supernatants were concentrated by centrifugation through an Amicon Ultracel 30K filter (EMD Millipore, Darmstadt, Germany). Plasma was isolated from whole blood using heparin vacutainer tubes (BD biosciences, San Jose, Calif.).

Exosome Isolation

Exosomes were isolated by serial ultracentrifugation (18). Exosomes were isolated from either the supernatants of 3 flasks of confluent RA FLS, 10 ml of plasma, or 1 ml of RA synovial fluid diluted 1:4 with PBS. Cells were pelleted out at 1500 rpm for 5 minutes. Then the supernatants were cleared of heavier debris by centrifugation at 10,000×g for 30 minutes and 30,000×g for 1 hour. Exosomes were then obtained by ultra-centrifugation at 110,000×g for 4-20 hours. Exosome pellets were washed in PBS at 110,000×g for 1.5 hours—overnight and resuspended in 1 ml of PBS. Some exosomes were further purified using a density gradient, Optiprep (Sigma Aldrich, St. Louis, Mo.). Optiprep was diluted with PBS to produce the following layers: 5%, 10%, 15%, 20%, 30%, 40%, and 50% w/v (densities of 1.031, 1.050, 1.084, 1.110, 1.163, 1.215, and 1.268 g/ml). 500 μl of extra-cellular vesicle fractions were floated on the top of the density gradient and the gradients were centrifuged at 100,000×g for 1 hour. Fractions were carefully pipetted off, washed with PBS, and centrifuged at 110,000×g for 2 hours. Pellets from the fractions were resuspended in 500 μl PBS. Exosome size was measured by use of a NanoSight NS500 (Malvern Instruments, Salisbury, United Kingdom).

CD13 Enzyme-Linked Immunosorbent Assay (ELISA)

High-binding ELISA plates were coated with the anti-CD13 monoclonal antibody WM15 (Biolegend, San Diego, Calif.) in 0.1M carbonate buffer pH 9.5 overnight, and then blocked with 1× Animal Free Block (Vector Laboratories, Burlingame, Calif.) overnight. Samples were then applied to the plates either whole or diluted in block with 10 mM EDTA. A standard curve was prepared using recombinant human CD13 (R&D Systems, Minneapolis, Minn.) in block with 10 mM EDTA. 1D7 (i.e., 591.1D7.34, University of Michigan Hybridoma Core), an anti-CD13 monoclonal antibody that was recently described (10), was biotinylated (Biotin-XX Microscale Protein Labeling Kit, Life Technologies, Carlsbad, Calif.) and applied overnight (19). Streptavidin-HRP (Biolegend, San Diego, Calif.) was then added. Between steps, plates were washed with PBS plus 0.05% Tween. The plates were visualized with TMB substrate (BD Biosciences, San Jose, Calif.), stopped with 2M H₂SO₄, and analyzed on a colorimetric plate reader.

ELISA for the Secretion of CD13 from RA FLS

To determine the role of tissue inhibitors of metalloproteinases (TIMPs) in the secretion of CD13, RA FLS (10 aliquots of 10⁵, or 10⁶, cells) were plated in 6-well plates in RPMI with 10% FBS. When RA FLS became 85% confluent, media was switched to RPMI containing 0.1% FBS. RA FLS were incubated with TIMP-1, TIMP-2 or both for 48 hours. ELISA was performed to determine the levels of soluble CD13 in the conditioned media. Conditioned media (100 μl/well) was added to 96-well plates (Thermo Scientific, USA) for 2 hours at room temperature. After blocking, anti-CD13 antibody, WM15, (10 μg/ml, BioLegend) was added for 2 hours at 37° C. Anti-mouse IgG HRP-linked antibody (1:1000) was added for 1 hour. After adding the TMB substrate solution (BD Biosciences) and stop solution (2N H2SO4), the optical density (OD) was measured at 450 nm by a microplate reader (BIO-TEK, USA). We used FLS from three different RA patients. Both inhibitors were used at 0.6 μg/ml. These concentrations are recommended by the manufacture to inhibit matrix metalloproteinases (MMPs).

Aminopeptidase Enzymatic Activity

Aminopeptidase activity was measured by cleavage of L-Leucine-7-amido-4-methyl coumarin (L-leu-AMC, Sigma-Aldrich, St. Louis, Mo.) to release the fluorescent molecule AMC. A standard curve was constructed using AMC (Sigma-Aldrich, St. Louis, Mo.). The assay was run in 0.1 M Tris-HCl buffer (pH 8.0). Samples were incubated with the substrate at 37° C. for one hour then read using a fluorescent plate reader at emission 450, excitation 365. Results were calculated as μM/hour of substrate cleaved.

Western Blot

Exosome lysates, derived from exosomes isolated from RA FLS (15 μl), were boiled for 5 minutes in Laemmli's sample buffer and subjected to 10% SDS-polyacrylamide gel electrophoresis (PAGE) followed by Western blot analysis. The proteins were electrophoretically transferred from the gel onto nitrocellulose membranes using a conventional Tris-glycine buffer. To block nonspecific binding, membranes were incubated with 5% nonfat milk in Tris-buffered saline containing 0.01% Tween-20 (TBST) for 1 hour at room temperature. The blots were incubated in mouse anti-human Flotillin and CD9 (BD Biosciences) primary antibodies plus 5% nonfat milk in TBST at 4° C. overnight. After washing with TBST, the blots were incubated with horseradish peroxidase-conjugated sheep anti-mouse and with goat anti-rabbit IgG (1:3000) for 45 minutes at room temperature. An ECL detection system was used to identify specific protein bands.

siRNA Knockdown

FLS were transfected by electroporation using an Amaxa Nucleofector and the nucleofector kit for dermal fibroblasts (NHDF, Lonza, Basel, Switzerland). In brief, FLS were released by trypsin and 5×10⁵ cells were transfected per condition. Cells were resuspended in transfection solution and either 300 nM MMP14 short inhibitory RNA (siRNA); MMP1 siRNA; ADAM15 siRNA, ADAM10 siRNA, ADAM17 siRNA (all, stealth RNAi (set of 3), Life Technologies, Carlsbad, Calif.); or 2 μg pmaxGFP (Lonza, Basel, Switzerland) was added to each transfection cuvette. Cells were electroporated and transferred to flasks containing 20% CMRL. Transfected cells were grown for 5-7 days and then transferred to serum-free medium for 2 days before harvesting. Transfection of GFP control plasmid was measured by fluorescent microscopy (EVOSfl, AMG, Mill Creek, Wash.) and flow cytometry (BD Biosciences FACSCalibur, San Jose, Calif.). Knockdown efficiency was measured by qRT-PCR of MMP14 mRNA at the time of harvest for CD13 measurements.

Confocal Microscopy

RA FLS were grown to 90% confluence on 8-well glass chamber slides. Cells were fixed with 1% Formalin and blocked with Fc block (10% human serum/10% mouse serum in PBS). Cells were incubated for 1 hour at room temperature with anti-CD13-FITC (1D7) at 1 μg/100 μl and anti-MMP14-PE (clone 128527, R&D, Minneapolis, Minn.) at 1.67 μg/100 μl or anti-CD90-PE at 1 μg/100 μl (3E10, Biolegend, San Diego, Calif.). All experiments also included staining with MsIgG isotype controls (MsIg-FITC (eBiosciences, San Diego, Calif.), MsIg-PE (Biolegend, San Diego, Calif.)) at the same concentrations. The nuclei were counter-stained with DAPI at 1 μg/ml. Cells were mounted using Pro-gold anti-fade media (Life Technologies, Carlsbad, Calif.). Images were taken with an Olympus Fluo-View 500 confocal microscope system (University of Michigan Microscopy and Image Analysis Core) at 600× and 1000×. All images were corrected for background, thresholds were determined by DAPI alone, MsIg-FITC alone, and MsIg-PE alone. Co-localization was analyzed with ImageJ using the plug-in “Colocalization”, by Pierre Bourdoncle, Institut Jacques Monod, Service Imagerie, Paris.

Quantitative RT-PCR

mRNA was isolated from FLS (3 wells of a 6-well plate) using the RNAeasy Kit and Qiacube (Qiagen, Venlo, Netherlands). cDNA was prepared using a High Capacity cDNA Kit (Life Technologies, Carlsbad, Calif.). Quantitative reverse transcription polymerase chain reaction (qRT-PCR) was done using TaqMan Gene Expression Assays on a 7500 Real Time PCR System (Life Technologies, Carlsbad, Calif.).

Flow Cytometry

Fibroblasts were removed from flasks by 3 mM EDTA in PBS. Cells were stained with MsIgG (negative control) or anti-CD13 (591.1D7.34), then goat anti-mouse IgG-Alexa fluor 488 (Life Technologies, Carlsbad, Calif.). Cytometry was performed on a BD Biosciences FACSCalibur. Gating was done to isolate the major cell population and exclude debris and dead cells.

FLS Growth and Migration Assays

RA FLS were seeded on Essen Image Lock 96-well plates (Essen Bioscience, Ann Arbor, Mich.) overnight at either 3,000 cells/well for growth or 30,000 cells/well for migration. For growth, the 20% FBS CMRL media was removed and cells were washed 1× with PBS. 100 μl of medium alone (control) or medium containing anti-CD3 (25 or 50 ng/ml, OKT3, used as a non-reactive isotype control antibody in this experiment), anti-CD13 1D7 or WM15 (Biolegend, San Diego, Calif.) (25 or 50 ng/ml), or CD13 chemical inhibitors actinonin (Sigma-Aldrich, St. Louis, Mo.) or bestatin (Sigma-Aldrich, St. Louis, Mo.) (10 μM and 50 μM, respectively) was added to the wells. Images and confluence data were collected using an Essen IncuCyte (Essen Bioscience, Ann Arbor, Mich.). For the scratch-wound migration assay, wounds were made via the Essen scratch-wound tool in the seeded 96-well plate. Plates were then washed 2× with PBS and medium was added similar to the growth plates. Data were collected and the confluence or relative wound density calculated by the Essen IncuCyte.

Statistics

Data are expressed as mean±standard error of the mean (SEM). FLS data are expressed as a ratio of treated FLS to untreated FLS. Statistical significance was determined by unpaired student T-test.

### Example 2

CD13 is found on Extracellular Vesicles Including Exosomes

CD13 is present in synovial fluids, serum, and FLS culture supernatants (10). The question remains, however, as to whether CD13 in those fluids is a soluble molecule or is bound on the surface of extracellular vesicles. To test the possibility that extracellular vesicles (EVs) also contain CD13, extracellular vesicles (EVs) were isolated and CD13 was measured in the EV and soluble-protein fractions. Differential ultracentrifugation was used to isolate EVs corresponding to the density of exosomes. CD13 was identified in both soluble protein and EV fractions in plasma, RA FLS culture supernatant, and RA synovial fluid (FIG. 1A). Plasma contained an average of 57.81±16.43 ng/ml of CD13 on EVs with an activity of 487.41±308.51 μM/hour, and 125.17±83.68 ng/ml of soluble CD13 with an activity of 2134.83±884.81 μM/hour. FLS culture supernatant contained an average of 41.89±35.76 ng/ml of CD13 on EVs with an activity of 241.00±137.01 μM/hour, and 39.71±11.91 ng/ml of soluble CD13 with an activity of 376.13±143.52 μM/hour. RA synovial fluid contained an average of 268.67±163.10 ng/ml of CD13 on EVs with an activity of 3327.33±3015.37 μM/hour, and 559.18±255.67 ng/ml of soluble CD13 with an activity of 3740.11±934.78 μM/hour. A significant difference was observed between the levels of CD13 (p=0.039) and aminopeptidase activity (p=0.012) in the soluble fractions of plasma compared to RA synovial fluid.

Although differential centrifugation is a suitable protocol for isolation of exosomes, there may be other contaminants of similar density (including apoptotic bodies and protein aggregates). Extracellular vesicles were therefore analyzed by NanoSight. Exosomes were defined as being of size 30-130 nm, which is consistent with the expected size of exosomes identified by NanoSight (20,21). Isolation of exosomes from FLS was confirmed by Western blot for flotillin-1 and CD9. Consistent with exosomes originating from FLS, a strong single band for flotillin-1 and a weak band for CD9 (22) were observed. Exosomes defined by size made up 71.8% of the EVs in RA FLS culture supernatant (mode 85.5 nm), 85.7% of the EVs in normal human plasma (mode 58.7 nm), and 57% of the EVs in RA synovial fluid (mode 110.1 nm). An example of the NanoSight data is provided in FIG. 6A. To further define which extracellular structures contain CD13, the EV fraction was divided by density over a discontinuous Optiprep density gradient with seven fractions from 1.268 g/ml to 1.031 g/ml. A band at the density gradient between fractions 4 and 5, where exosomes would be expected (density between 1.084 g/ml and 1.163 g/ml fractions 3-5), was visually confirmed. ELISA also revealed CD13 in all seven gradient fractions, as well as in soluble protein fractions (FIG. 6B). The three types of fluids that were analyzed (i.e., RA FLS culture supernatant, healthy control plasma, and RA synovial fluid) each exhibited a distinct pattern of CD13 localization (FIG. 1C). FLS culture supernatant was the most balanced with an average of 48.67% soluble CD13, 29.76% exosomal CD13, and 21.58% other EV CD13. Healthy control plasma was predominantly soluble with 85.60% soluble CD13, 7.09% exosomal CD13, and 7.31% CD13 on other EVs. The RA synovial fluid contained 67.55% soluble CD13, 11.92% exosomal CD13, and 20.53% CD13 from other EVs.

### Example 3

Metalloproteinases Cleave CD13 from FLS

Because CD13 exists as a soluble molecule in cell-free portions of biological fluids separate from vesicle-associated CD13, soluble CD13 must be released from cells. Because soluble CD13 was found in FLS culture supernatants, the release of CD13 from FLS was explored. CD13 is highly expressed on the cell surface of FLS and therefore could be shed. To test this mechanism, various protease inhibitors were added to FLS cultures, including: pepstatin A (aspartic), aprotinin (serine), leupeptin (serine/cysteine), GM6001 (metalloproteinase), and E-64 (cysteine). Only one, GM6001, was found to block CD13 release from FLS. All inhibitors were used at established working concentrations. All of these inhibitors are known to have low toxicity, and no significant cell death was observed, as measured by trypan blue staining upon culture harvest. Therefore, pharmacologic toxicity did not contribute to the results (23,24). In all cases, total CD13 concentration was higher in the cell lysate than in the cell culture supernatant. GM6001 significantly reduced CD13 protein found in the supernatant by 93.62±4.78%, p≤0.0001 (FIG. 2A). Leupeptin led to a significant (p≤0.05) increase (48.40±14.29%) in CD13 released. To confirm that the inhibitors were affecting cleavage and not expression, CD13 was also measured in the FLS cell lysates. No significant difference was observed with GM6001; however, aprotinin induced a significant increase (22.17±6.18%, p≤0.05) in CD13 expression (FIG. 2B). These results indicate that CD13 is cleaved from FLS by metalloproteinases.

Tissue inhibitors of metalloproteinases (TIMPs) were used to determine whether CD13 was cleaved by soluble versus membrane-bound matrix metalloproteinases. A significant decrease in CD13 secretion was found in the conditioned media collected from RA FLS incubated with TIMP-2 for 48 hours (p<0.05). This decrease was not observed when RA FLS were incubated with TIMP-1, indicating that TIMP-2 contributes to the secretion of CD13 from FLS (FIG. 2C). This shows that a membrane-type matrix metalloproteinase is involved in the cleavage of CD13.

### Example 4

MMP14/MT1-MMP Cleaves CD13 from FLS

Matrix metalloproteinase 14 (MMP14/MT1-MMP) is a membrane-type metalloproteinase on FLS that is critical to FLS invasion of collagenous structures (25,26). MMP14 is up-regulated on RA FLS, so an investigation was undertaken to determine whether the metalloproteinase that cleaves CD13 from FLS is MMP14 (26-28). The investigation took the form of knocking down the expression of MMP14 in RA FLS using siRNA. FIG. 3A shows an example of the successful knockdown of MMP14. The fold-change for MMP14 over GAPDH and the ratio to mock control (ΔΔCt) of FLS was 1.10±0.10, and was decreased to 0.13±0.0064 with addition of MMP14 siRNA. ADAM15 siRNA, ADAM10 siRNA, ADAM17 siRNA also significantly knocked down the expression of their respective mRNAs (FIG. 3A). Moreover, no off-target effects were seen on MMP1 mRNA. GFP plasmid transfection was used to determine transfection efficiency. Higher fluorescence was observed with both flow cytometry and fluorescent microscopy over mock transfection controls. Green fluorescence measured by flow cytometry increased from 6.77 mean fluorescent intensity (MFI) in the negative control (mock transfected) to 111.23 MFI in the transfected FLS.

Knockdown (KD) of MMP14 significantly decreased the CD13 released from FLS (FIG. 3B). Samples were normalized to mock transfection in order to compare between experiments (n≥3) and CD13 in mock transfection is shown on the graph as a line at one. The only significant difference in the siRNA KD of test groups (MMP14, ADAM15, ADAM17, ADAM10) was seen with MMP14 (n=14), in which case the KD cells released CD13 at 0.78±0.08 of control levels, p=0.0031 (FIG. 3B). CD13KD was used as a positive control, and MMP1 KD was used as a negative control for membrane-anchored metalloproteinases. To confirm cleavage was being measured and not a decrease in CD13 expression, CD13 protein was also measured in cell lysates. Knockdown of MMP14 did not significantly alter cellular CD13; MMP14 KD ratio over mock was 1.24±0.25 (FIG. 3B). Because MMP14 KD resulted in an average decrease of only 23% in supernatant CD13, this indicates that more than one protease can cleave CD13. To confirm that the other CD13-releasing protease(s) are also metalloproteinases, GM6001 was added to MMP14 KD cultures (FIG. 3B, n=2). Similar to previous results, GM6001 prevented the release of CD13 into the culture supernatant (p=1.42×10⁻⁹). Addition of GM6001 to MMP14 KD cultures also decreased supernatant CD13 to a level significantly lower than MMP14 KD without GM6001 (p=3.21×10⁻⁹). In the lysates of GM6001-treated cells, CD13 was significantly increased (p=0.0070). Single knockdown of several other possible candidates (ADAM15, ADAM10, or ADAM17) did not decrease CD13 release from FLS, and ADAM17 KD did not add to the effect of MMP14 KD. ADAM17 KD actually led to a significant increase in CD13 expression in both the cell lysate and culture supernatant (p=2.01×10⁻⁸ and p=0.028, respectively). It should be noted that siRNA KD of CD13 itself (n=2) resulted in an approximate 50% decrease relative to mock transfected cell lysate (0.40±0.031, p=1.18×10⁻¹⁷) or culture supernatant (0.50±0.0083, p=7.58×10⁻²⁶). This shows that additional metalloproteinases, in addition to MMP14, are involved in the cleavage of CD13, but does not identify any one enzyme of equivalent importance to MMP14 in the shedding of CD13.

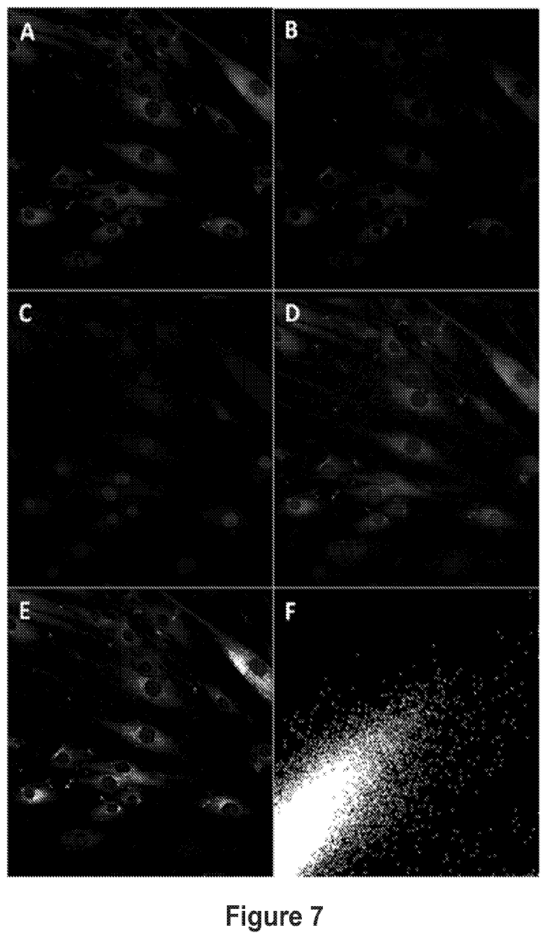

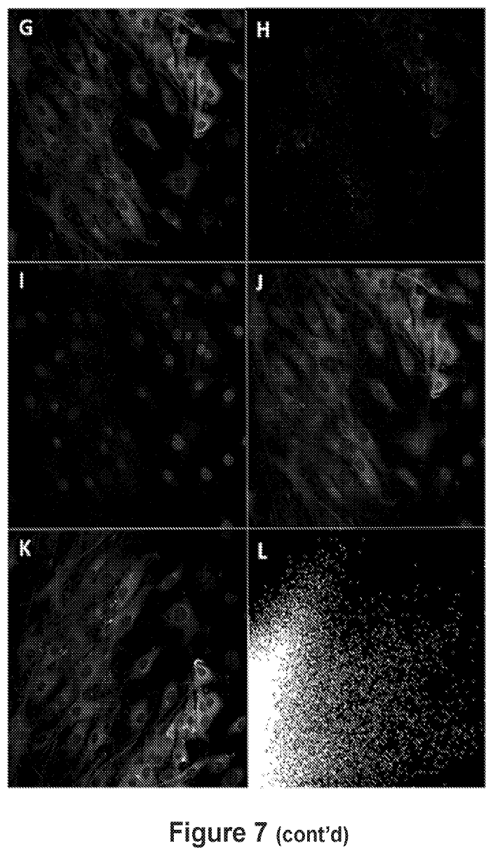

To confirm the role of MMP14 in the release of CD13, confocal microscopy was used to look for co-localization on the surface of RA FLS. Cells were stained with DAPI for nuclei (blue, not shown), anti-CD13-FITC (green), and anti-MMP14-PE (red). Predominant co-localization of CD13 and MMP14 was observed on all tested FLS, with lesser areas of individual staining. Images shown in FIG. 3C are representative of n=6. The bottom left-hand picture shows CD13 (green) and MMP14 (red) signals overlapping on FLS to form yellow. The bottom right-hand picture shows a computer analysis, with co-localized pixels in white, and individual color areas in green or red. Staining of a second FLS line is shown in FIG. 7 with a CD90 control. The results indicate that MMP14 co-localizes with CD13 on FLS and contributes to cleaving of CD13 from the FLS membrane to generate its soluble form. Other undetermined metalloproteinases (likely redundantly and in combination) also contribute to CD13 cleavage.

### Example 5

Regulation of CD13 Expression on FLS

CD13 is present at much higher levels in RA synovial fluid compared to OA. Cultured RA and OA FLS, however, expressed similar amounts of CD13 (10). One possible explanation for this observation is that the pro-inflammatory cytokines in the RA joint could contribute to upregulation of CD13 production by FLS. Cultured RA FLS were treated with IFNγ, TNFα, or IL-17 over a time course from 0 to 72 hours. CD13 mRNA, as measured by qRT-PCR, was upregulated by all three cytokines with a peak expression around 48 hours (FIG. 4A). Data shown are a ratio of cytokine-treated FLS CD13 mRNA to untreated FLS CD13 mRNA at the same time point. IFNγ- and TNFα-exposed cells were significantly upregulated (p≤0.05) at 12, 24, 48 and 72 hours, and the IL-17 effect was significant at 8, 12, 24, and 48 hours. Expression of CD13 protein, however, exhibited variability and fluctuations that did not match the change in CD13 mRNA. FIG. 4B shows the results of one cell line examined by flow cytometry with staining by anti-CD13 (1D7). FIG. 4C shows CD13 (ng/ml) in total cell lysate, and FIG. 4D shows CD13 (ng/ml) in FLS cell supernatant (both measured by ELISA). Other FLS lines showed comparable fluctuations, which may in part be explained by shifting of CD13 between various cellular and extracellular compartments.

### Example 6

CD13 Aids in Growth and Migration of RA FLS

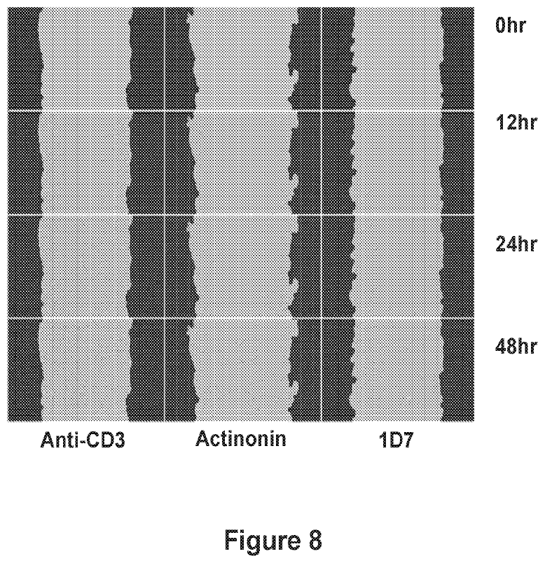

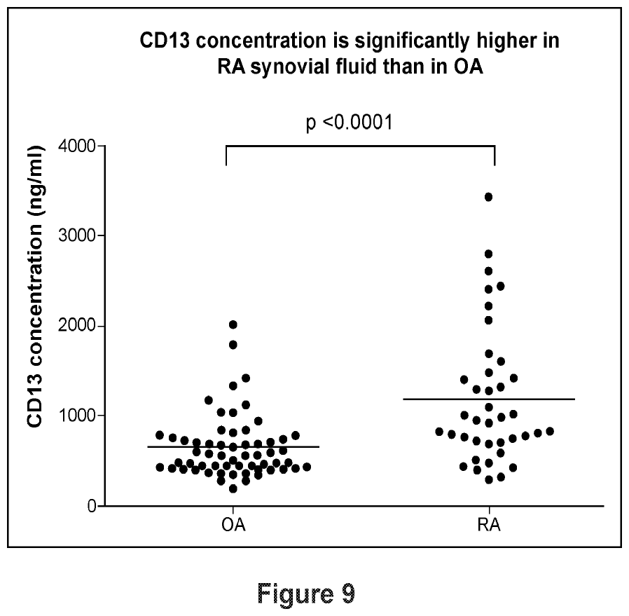

One mechanism that could account for fluctuating levels of soluble CD13 in FLS culture media would be uptake of CD13 by the RA FLS in an autocrine manner. To determine possible functions for CD13 on FLS, the effect of anti-CD13 antibodies (WM15 or 1D7) or CD13 chemical inhibitors (Bestatin or Actinonin) on RA FLS growth and migration was examined. Anti-CD3 was used as a negative, isotype control. A significant slowing of cell growth was observed with both CD13 inhibitors and with both anti-CD13 antibodies (FIG. 5A). Data are expressed as the change from 0 hour as a ratio to untreated FLS of the same cell line at the same time point. The significant (p≤0.05) slowing of growth was observed primarily between 24 hours and 120 hours with actinonin being the strongest inhibitor of cell proliferation. A significant decrease (p≤0.05) was also seen in RA FLS migration in a wound healing assay in the presence of actinonin, WM15 or 1D7, primarily from 36 hours to 72 hours (FIG. 5B). These data demonstrate an autocrine effect of CD13 on FLS. Examples of scratch-wound images for anti-CD3 control, actinonin, and 1D7 are shown in FIG. 8.

Each of the references listed immediately below and cited throughout the disclosure is incorporated by reference herein in its entirety, or in relevant part, as would be apparent from context.

### REFERENCES (OTHER THAN FOR EXAMPLES 7-9)

1. Look A T, Ashmun R A, Shapiro L H, Peiper S C. Human myeloid plasma membrane glycoprotein CD13 (gp150) is identical to aminopeptidase N. J Clin Invest. 1989 April; 83(4): 1299-307 .
2. Luan Y, Xu W. The structure and main functions of aminopeptidase N. Curr Med Chem. 2007; 14(6):639-47.
3. Mina-Osorio P. The moonlighting enzyme CD13: old and new functions to target. Trends Mol Med. 2008 August; 14(8):361-71.
4. Riemann D, Kehlen A, Langner J. CD13—not just a marker in leukemia typing. Immunol Today. 1999 February; 20(2):83-8.
5. Dan H, Tani K, Hase K, Shimizu T, Tamiya H, Biraa Y, et al. CD13/aminopeptidase N in collagen vascular diseases. Rheumatol Int. 2003 November; 23(6):271-6.
6. Shimizu T, Tani K, Hase K, Ogawa H, Huang L, Shinomiya F, et al. CD13/aminopeptidase N-induced lymphocyte involvement in inflamed joints of patients with rheumatoid arthritis. Arthritis Rheum. 2002 September; 46(9):2330-8.
7. Kitko C L, Levine J E, Storer B E, Chai X, Fox D A, Braun T M, et al. Plasma CXCL9 elevations correlate with chronic GVHD diagnosis. Blood. 2014 Jan. 30; 123(5):786-93.
8. Thielitz A, Ansorge S, Bank U, Tager M, Wrenger S, Gollnick H, et al. The ectopeptidases dipeptidyl peptidase IV (DPIV) and aminopeptidase N (APN) and their related enzymes as possible targets in the treatment of skin diseases. Front Biosci J Virtual Libr. 2008; 13:2364-75.
9. Nefla M, Sudre L, Denat G, Priam S, Andre-Leroux G, Berenbaum F, et al. The pro-inflammatory cytokine 14-3-3ε is a ligand of CD13 in cartilage. J Cell Sci. 2015 Sep. 1; 128(17):3250-62.
10. Morgan R, Endres J, Behbahani-Nejad N, Phillips K, Ruth J H, Friday S C, et al. Expression and Function of Aminopeptidase N/CD13 Produced by Fibroblast-like Synoviocytes in Rheumatoid Arthritis: Role of CD13 in Chemotaxis of Cytokine-Activated T Cells Independent of Enzymatic Activity. Arthritis Rheumatol Hoboken N.J. 2015 January; 67(1):74-85.
11. Watanabe Y, Ito K, Iwaki-Egawa S, Yamaguchi R, Fujimoto Y. Aminopeptidase N in sera of healthy subjects is a different N-terminal processed derivative from the one obtained from maternal serum. Mol Genet Metab. 1998 April; 63(4):289-94.
12. Kawai M, Hara Y, Miyazato I, Hosaki S. Novel, aberrantly truncated isoform of serum CD13 in a family with high serum aminopeptidase N (CD13) activity. Clin Chem. 2001 February; 47(2):223-30.
13. E L Andaloussi S, Mager I, Breakefield X O, Wood MJA. Extracellular vesicles: biology and emerging therapeutic opportunities. Nat Rev Drug Discov. 2013 May; 12(5):347-57.
14. Simpson R J, Jensen S S, Lim J W E. Proteomic profiling of exosomes: current perspectives. Proteomics. 2008 October; 8(19):4083-99.
15. Thery C, Zitvogel L, Amigorena S. Exosomes: composition, biogenesis and function. Nat Rev Immunol. 2002 August; 2(8):569-79.
16. Zimmermann T, Kunisch E, Pfeiffer R, Hirth A, Stahl H D, Sack U, et al. Isolation and characterization of rheumatoid arthritis synovial fibroblasts from primary culture—primary culture cells markedly differ from fourth-passage cells. Arthritis Res. 2001; 3(1):72-6.
17. Arnett F C, Edworthy S M, Bloch D A, McShane D J, Fries J F, Cooper N S, et al. The American Rheumatism Association 1987 revised criteria for the classification of rheumatoid arthritis. Arthritis Rheum. 1988 March; 31(3):315-24.
18. Thery C, Amigorena S, Raposo G, Clayton A. Isolation and characterization of exosomes from cell culture supernatants and biological fluids. Curr Protoc Cell Biol Editor Board Juan Bonifacino Al. 2006 April; Chapter 3:Unit 3.22.
19. Bouchet S, Tang R, Fava F, Legrand O, Bauvois B. Targeting CD13 (aminopeptidase-N) in turn downregulates ADAM17 by internalization in acute myeloid leukaemia cells. Oncotarget. 2014 Sep. 30; 5(18):8211-22.
20. Sokolova V, Ludwig A-K, Hornung S, Rotan O, Horn P A, Epple M, et al. Characterisation of exosomes derived from human cells by nanoparticle tracking analysis and scanning electron microscopy. Colloids Surf B Biointerfaces. 2011 Oct. 1; 87(1):146-50.
21. Caradec J, Kharmate G, Hosseini-Beheshti E, Adomat H, Gleave M, Guns E. Reproducibility and efficiency of serum-derived exosome extraction methods. Clin Biochem. 2014 September; 47(13-14):1286-92.
22. Kato T, Miyaki S, Ishitobi H, Nakamura Y, Nakasa T, Lotz M K, et al. Exosomes from IL-1β stimulated synovial fibroblasts induce osteoarthritic changes in articular chondrocytes. Arthritis Res Ther. 2014; 16(4):R163.
23. Overall C M, Blobel C P. In search of partners: linking extracellular proteases to substrates. Nat Rev Mol Cell Biol. 2007 March; 8(3):245-57.
24. Grinde B, Seglen P O. Differential effects of proteinase inhibitors and amines on the lysosomal and non-lysosomal pathways of protein degradation in isolated rat hepatocytes. Biochim Biophys Acta BBA—Gen Subj. 1980 Sep. 17; 632(1):73-86.
25. Sabeh F, Li X-Y, Saunders T L, Rowe R G, Weiss S J. Secreted versus membrane-anchored collagenases: relative roles in fibroblast-dependent collagenolysis and invasion. J Biol Chem. 2009 Aug. 21; 284(34):23001-11.
26. Sabeh F, Fox D, Weiss S J. Membrane-type I matrix metalloproteinase-dependent regulation of rheumatoid arthritis synoviocyte function. J Immunol Baltim Md. 1950.2010 Jun. 1; 184(11):6396-406.
27. Jain A, Miller M-C, Troeberg L, Itoh Y, Brennan F, Nanchahal J. Invasive potential of human rheumatoid tenosynovial cells is in part MT1-MMP dependent. J Hand Surg. 2009 September; 34(7):1282-90.
28. Seemayer C A, Kuchen S, Kuenzler P, Rihoskova V, Rethage J, Aicher W K, et al. Cartilage destruction mediated by synovial fibroblasts does not depend on proliferation in rheumatoid arthritis. Am J Pathol. 2003 May; 162(5):1549-57.
29. Favaloro E J, Browning T, Facey D. CD13 (GP150; aminopeptidase-N): predominant functional activity in blood is localized to plasma and is not cell-surface associated. Exp Hematol. 1993 December; 21(13):1695-701.
30. Van Hensbergen Y, Broxterman H J, Hanemaaijer R, Jorna A S, van Lent N A, Verheul HMW, et al. Soluble aminopeptidase N/CD13 in malignant and nonmalignant effusions and intratumoral fluid. Clin Cancer Res Off J Am Assoc Cancer Res. 2002 December; 8(12):3747-54.
31. Arpino V, Brock M, Gill S E. The role of TIMPs in regulation of extracellular matrix proteolysis. Matrix Biol. 2015 May; 44-46:247-54.
32. Yamanaka H, Makino K, Takizawa M, Nakamura H, Fujimoto N, Moriya H, et al. Expression and tissue localization of membrane-types 1,2, and 3 matrix metalloproteinases in rheumatoid synovium. Lab Investig J Tech Methods Pathol. 2000 May; 80(5):677-87.
33. Pap T, Shigeyama Y, Kuchen S, Fernihough J K, Simmen B, Gay R E, et al. Differential expression pattern of membrane-type matrix metalloproteinases in rheumatoid arthritis. Arthritis Rheum. 2000 June; 43(6):1226-32.
34. Miller M-C, Manning H B, Jain A, Troeberg L, Dudhia J, Essex D, et al. Membrane type 1 matrix metalloproteinase is a crucial promoter of synovial invasion in human rheumatoid arthritis. Arthritis Rheum. 2009 March; 60(3):686-97.
35. Marco M, Fortin C, Fulop T. Membrane-type matrix metalloproteinases: key mediators of leukocyte function. J Leukoc Biol. 2013 August; 94(2):237-46.
36. Van der Vorst E P C, Keijbeck A A, de Winther M P J, Donners M M P C. A disintegrin and metalloproteases: Molecular scissors in angiogenesis, inflammation and atherosclerosis. Atherosclerosis. 2012 October; 224(2):302-8.
37. Khokha R, Murthy A, Weiss A. Metalloproteinases and their natural inhibitors in inflammation and immunity. Nat Rev Immunol. 2013 September; 13(9):649-65.
38. Klein T, Bischoff R. Active metalloproteases of the A Disintegrin and Metalloprotease (ADAM) family: biological function and structure. J Proteome Res. 2011 Jan. 7; 10(1):17-33.
39. Dreymueller D, Pruessmeyer J, Groth E, Ludwig A. The role of ADAM-mediated shedding in vascular biology. Eur J Cell Biol. 2012 June; 91(6-7):472-85.
40. Le Gall S M, Bobé P, Reiss K, Horiuchi K, Niu X-D, Lundell D, et al. ADAMs 10 and 17 represent differentially regulated components of a general shedding machinery for membrane proteins such as transforming growth factor alpha, L-selectin, and tumor necrosis factor alpha. Mol Biol Cell. 2009 March; 20(6):1785-94.
41. Saftig P, Reiss K. The “A Disintegrin And Metalloproteases” ADAM10 and ADAM17: novel drug targets with therapeutic potential? Eur J Cell Biol. 2011 July; 90(6-7):527-35.
42. Bohm B B, Freund I, Krause K, Kinne R W, Burkhardt H. ADAM15 adds to apoptosis resistance of synovial fibroblasts by modulating focal adhesion kinase signaling. Arthritis Rheum. 2013 November; 65(11):2826-34.
43. Gao J, Zheng W, Wang L, Song B. A disintegrin and metallproteinase 15 knockout decreases migration of fibroblast-like synoviocytes and inflammation in rheumatoid arthritis. Mol Med Rep. 2015 Feb. 4;
44. Komiya K, Enomoto H, Inoki I, Okazaki S, Fujita Y, Ikeda E, et al. Expression of ADAM15 in rheumatoid synovium: up-regulation by vascular endothelial growth factor and possible implications for angiogenesis. Arthritis Res Ther. 2005; 7(6):R1158-73.
45. Hawari F I, Rouhani F N, Cui X, Yu Z-X, Buckley C, Kaler M, et al. Release of full-length 55-kDa TNF receptor 1 in exosome-like vesicles: a mechanism for generation of soluble cytokine receptors. Proc Natl Acad Sci USA. 2004 Feb. 3; 101(5):1297-302.
46. Rozanov D V, Deryugina E I, Monosov E Z, Marchenko N D, Strongin A Y. Aberrant, persistent inclusion into lipid rafts limits the tumorigenic function of membrane type-1 matrix metalloproteinase in malignant cells. Exp Cell Res. 2004 Feb. 1; 293(1):81-95.
47. Riemann D, Hansen G H, Niels-Christiansen L, Thorsen E, Immerdal L, Santos A N, et al. Caveolae/lipid rafts in fibroblast-like synoviocytes: ectopeptidase-rich membrane microdomains. Biochem J. 2001 Feb. 15; 354(Pt 1):47-55.
48. Mathivanan S, Ji H, Simpson R J. Exosomes: extracellular organelles important in intercellular communication. J Proteomics. 2010 Sep. 10; 73(10):1907-20.
49. Tran C N, Lundy S K, White P T, Endres J L, Motyl C D, Gupta R, et al. Molecular interactions between T cells and fibroblast-like synoviocytes: role of membrane tumor necrosis factor-alpha on cytokine-activated T cells. Am J Pathol. 2007 November; 171(5):1588-98.
50. Tran C N, Thacker S G, Louie D M, Oliver J, White P T, Endres J L, et al. Interactions of T cells with fibroblast-like synoviocytes: role of the B7 family costimulatory ligand B7-H3. J Immunol Baltim Md. 1950. 2008 Mar. 1; 180(5):2989-98.
51. Kim K-W, Cho M-L, Kim H-R, Ju J-H, Park M-K, Oh H-J, et al. Up-regulation of stromal cell-derived factor 1 (CXCL12) production in rheumatoid synovial fibroblasts through interactions with T lymphocytes: role of interleukin-17 and CD40L-CD40 interaction. Arthritis Rheum. 2007 April; 56(4):1076-86.
52. Licona-Limón I, Garay-Canales C A, Mu\hat{n}oz-Paleta O, Ortega E. CD13 mediates phagocytosis in human monocytic cells. J Leukoc Biol. 2015 July; 98(1):85-98.
53. Villase\hat{n}or-Cardoso M I, Frausto-Del-Rio D A, Ortega E. Aminopeptidase N (CD13) is involved in phagocytic processes in human dendritic cells and macrophages. BioMed Res Int. 2013; 2013:562984.
54. Ghosh M, Subramani J, Rahman M M, Shapiro L H. CD13 restricts TLR4 endocytic signal transduction in inflammation. J Immunol Baltim Md. 1950. 2015 May 1; 194(9):4466-76.
55. Navarrete Santos A, Roentsch J, Danielsen E M, Langner J, Riemann D. Aminopeptidase N/CD13 is associated with raft membrane microdomains in monocytes. Biochem Biophys Res Commun. 2000 Mar. 5; 269(1):143-8.
56. Lai A, Ghaffari A, Ghahary A. Inhibitory effect of anti-aminopeptidase N/CD13 antibodies on fibroblast migration. Mol Cell Biochem. 2010 October; 343(1-2):191-9.
57. Petrovic N, Schacke W, Gahagan J R, O'Conor C A, Winnicka B, Conway R E, et al. CD13/APN regulates endothelial invasion and filopodia formation. Blood. 2007 Jul. 1; 110(1):142-50.

### Example 7

CD13 Aids in Growth and Migration of RA FLS

Determination of the Contribution of CD13 in RA Angiogenesis.

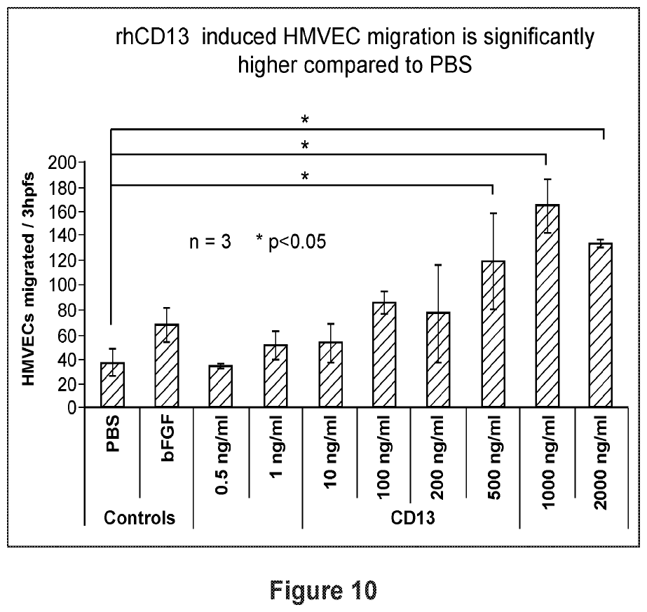

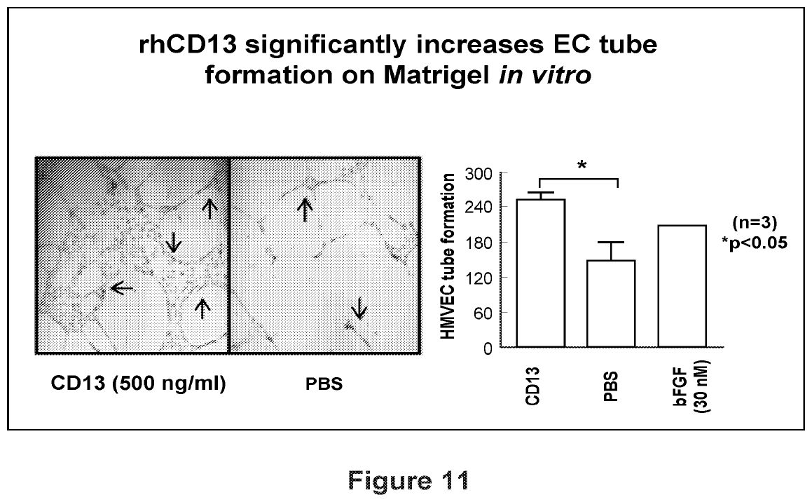

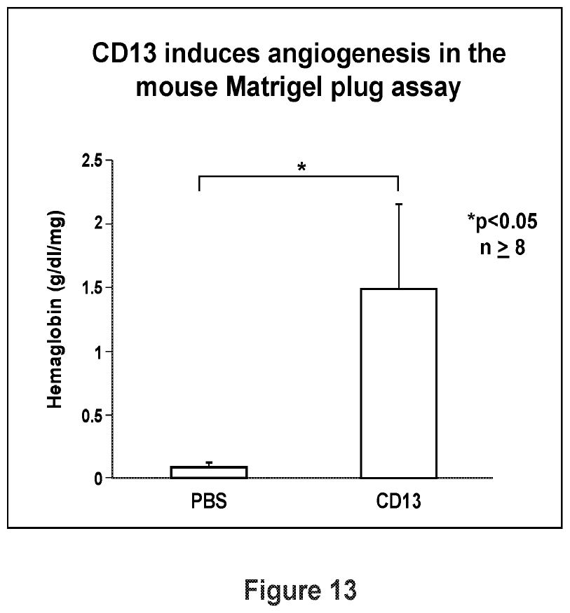

Rheumatoid arthritis (RA) is a chronic inflammatory disorder characterized by progressive joint destruction that affects 0.5-1% of adults in developed countries (52-54). Angiogenesis, or new blood vessel formation, plays an important role in vasculoproliferative diseases and is one of the earliest pathologic processes seen in RA (12-14, 55). Angiogenesis contributes to the growth and proliferation of ST by providing nutrients and a pathway for mononuclear cell ingress. It is a crucial step in pannus formation which causes cartilage and bone destruction (12-14). Strong data indicates that CD13 plays an important role in angiogenesis in vitro and in vivo (FIGS. 10, 11, 13). CD13 is higher in RA SFs than in OA and is highly expressed on RA FLS, indicating the importance of CD13 in RA. The importance of CD13 in RA is evaluated by performing a number of angiogenesis assays using RA SFs depleted of CD13.

Whether RA SF CD13 Depletion or Neutralization Leads to Less HMVEC Tube Formation on Matrigel

Immunodepletion of CD13 in RA SFs. SFs are collected from RA patients during therapeutic arthrocentesis with Institutional Review Board approval. RA SFs (6-8) are pooled and preincubated with 25 μg/ml of anti-CD13 from Biolegend or mouse IgG control to immunodeplete CD13 for 1 hour.

HMVEC Matrigel tube formation assay. After finding that CD13 is chemotactic for HMVECs, the role of CD13 in RA is examined by performing HMVEC capillary morphogenesis assay on Matrigel tube with sham- or CD13-depleted RA SFs. Growth factor-reduced Matrigel (GFR, BD Biosciences) is used to perform this assay (56-58). CD13- or sham-depleted RA SFs are used as test groups (58). The chambers are incubated for 16 to 18 hours at 37° C. in a 5% CO₂ humidified atmosphere. Cells are fixed and stained with Diff-Quik. The number of tubes formed is quantitated by an observer blinded to the experimental groups. bFGF (30 nM) and phosphate buffered saline (PBS) serve as positive and negative controls, respectively.

Whether RA SF CD13 Depletion or Neutralization Leads to Reduced 3D Spheroid EC Sprouting:

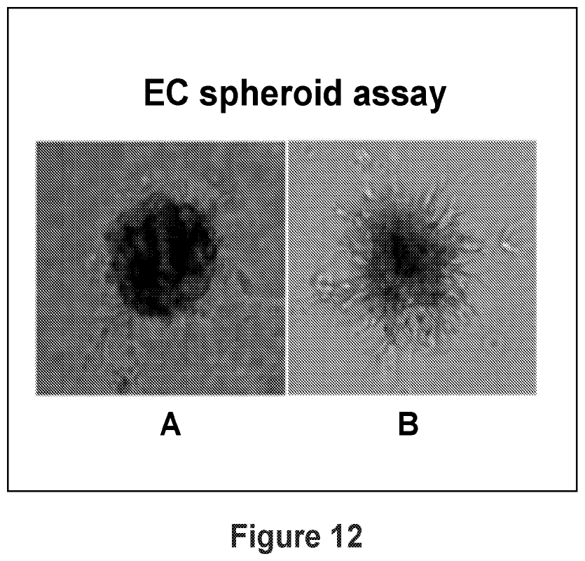

This assay involves the interaction of ECs and FLS and results in 3D EC sprouting (59, 60). This interaction plays an essential role in the proliferation and survival of both cells in RA. HMVECs and RA FLS will be harvested and fluorescently dye-tagged for visualization using Qtracker labeling kits (Thermo Fisher Scientific). Co-cultures of HMVECs (50,000) and RA ST FLS (25,000) are suspended in a mixture of 1 volume of 1.2% (wt/wt) methylcellulose (Sigma Aldrich) and 4 volumes of endothelial culture medium containing 5% fetal bovine serum (FBS) and antibiotics. This cell mixture is plated in U-bottom 96-well plates to facilitate spheroid formation as shown in FIG. 12. After 24 hours of incubation at 37° C., spheroids are resuspended in GFR Matrigel (diluted 1:5 with PBS) and plated on Matrigel-layered 96-well plates. Sham- or CD13-depleted RA SFs are used as stimuli, while bFGF or PBS serve as positive and negative controls, respectively. The media is switched every day. Sprouts are observed every day via both light microscopy and fluorescent microscopy for 3-5 days. The sprouts are analyzed and counted by two independent observers blinded to experimental groups. The number and length of the sprouts correlates to the angiogenicity of the tested substance (59, 60).

Whether Depletion of CD13 from RA SFs Results in Decreased Angiogenesis in the Mouse Matrigel Plug Assay.

This assay is performed as described previously (56-58). RA SFs sham- or CD13-depleted (50 μl in 500 μl of Matrigel) are injected subcutaneously in C57Bl/6 mice. After 7 days, the mice are euthanized and the Matrigel plugs are removed, weighed, and processed for hemoglobin (Hb) measurement or immunofluorescence. Hemoglobin (Hb) concentration is determined either by the Drabkin method using a Drabkin's reagent kit (Sigma) or with the 3,3,5,5-tetramethylbenzidine substrate system (Sigma) (56, 61, 62). Hb values are normalized to plug weight.

Immunofluorescence to detect angiogenesis in the Matrigel plugs. Some of the plugs will be embedded in OCT, cryosectioned, and immunofluorescence performed using rabbit anti-mouse vWF antibody (57). Blood vessels are quantitated by an observer blinded to the experimental groups.

Whether RA SFs Depleted of CD13 Induce Less HMVEC Homing into RA ST-SCID Mouse Chimera.

This is a novel angiogenic assay in which dye-tagged mature human HMVECs are injected into SCID mice, and their homing/angiogenesis is determined in the RA ST-SCID mouse chimera. This assay has shown that mature HMVECs recruitment or homing is significantly higher into RA STs with an angiogenic stimulus (63). SCID mice (6-8-week-old female mice) are anesthetized, shaved and grafted subcutaneously with ST (0.5 cm3 in size) in the backs of the animals. Each ST/mouse is implanted and the wound is sutured. After 3-4 weeks of engraftment, HMVECs (2×10⁶) dye-tagged with PKH26 (Sigma Aldrich, St Louis, Mo.) is injected intravenously into the SCID mice. At the same time, RA SFs CD13- or sham-depleted (100 μl/graft) are injected into some of the ST grafts, while other grafts receive PBS (a negative control). The grafts are harvested after 2 weeks of HMVEC injection and snap-frozen in OCT (Miles, Elkhart, Ind.). One section from each mouse is counted by an observer blinded to the test groups. Immunofluorescence will also be performed with cryosections using mouse anti-human CD31 antibody (BD Biosciences, San Jose, Calif.) to detect HMVECs migration and incorporation into new blood vessel formation in response to sham- or CD13-depleted SFs.

Whether Neutralization of CD13 Results in Less FLS-Induced Angiogenesis and Cartilage Invasion in Chick Chorioallantoic Membrane (CAM) Assay.

RA FLS-initiated angiogenic responses play a major role in the invasion of cartilage and joint destruction, providing the nutrients for FLS proliferation and the ingress of inflammatory cells into ST. CD13 has been shown to be a potent angiogenic factor and is secreted by RA FLS (FIG. 1) (1). In this assay, it is determined whether knocking down CD13 in RA FLS results in less angiogenesis and cartilage invasion. It has been shown that invasion of cartilage by FLS is dependent on MT1-MMP/MMP-14 (64), an FLS cell membrane protease that also releases sCD13 from the FLS surface.

FLSs are prepared after digesting RA STs with a solution of Dispase, collagenase, and DNase, as described (65). The FLS are used at passage 3 or later, at which time they are a homogeneous population. RA FLS are transfected with CD13 siRNA or control siRNA (Santa Cruz) using a nucleofector kit and electroporation (Amaxa Biosystems; Koln, Germany). CD13 siRNA- or control siRNA-transfected FLS (5×10⁵ cells) are labeled with dioctadecyloxacarbocyanine perchlorate (DiO; Molecular Probes). Articular cartilage fragments dissected from the knee joints of white rabbits are cultured with RA FLS transfected with CD13 siRNA or control siRNA for 2 hours in vitro to allow the cells to adhere to the tissue explants. FLS-cartilage co-cultures are placed atop the CAM of 11-day-old chick embryos (Chick embryos are very angiogenic at this stage) for 4 days (64). The invasive activities of RA FLS is analyzed in cross sections of the recovered cartilage fragments. Fluorescence images of cell invasion into cartilage fragments or the CAM is captured with a Spot digital camera (Diagnostic Instruments, Sterling Heights, Mich.) through an upright microscope (Leica Microsystems, Deerfield, Ill.). In the immuno-incompetent setting of the chick embryo, RA FLS transfected with control siRNA should rapidly invade the cartilage matrix during the 4 day culture period. FLS transfected with MMP-14 siRNA is expected to be unable to invade cartilage (64). By contrast, FLS transfected with CD13 siRNA is expected to show less cartilage invasion, and much less angiogenesis on CAM.

We have found that CD13 plays an important role in angiogenesis in vitro and in vivo, as shown by the data disclosed herein. It is expected that a more than 2-3 fold decrease in angiogenesis assays in vitro and in vivo performed with RA SFs depleted of CD13 and less invasion of the cartilage is expected with RA FLS transfected with CD13 siRNA. Purified anti-human CD13 antibody (WM15) from Biolegend is used, as it has been used for a number of assays in our laboratory (1), and, alternatively, the 1D7 anti-CD13 monoclonal antibody (mAb) produced in our laboratory (1). We also have used this antibody in our assays and found that it neutralizes CD13 induced MN migration in vitro. It is not easy to transfect primary cells like FLS using routine transfection reagents. Electroporation-based methods are used to transfect FLS using nucleofector kit (Amaxa Biosystems; Koln, Germany). PCR is also performed to ensure that CD13 is knocked down after electroporation before performing a CAM assay. A 3-4 fold increase in angiogenesis is expected with sham-depleted RA SFs compared to CD13-depleted RA SFs. No difficulty is expected in using the ST-SCID chimera model with HMVECs or other assays. Most of the assays have been shown to function well, as shown by the data disclosed herein (56, 61-63).

Because CD13 is a multifunctional protein with both N-aminopeptidase activity and G-protein coupled receptor (GPCR) binding properties (1), the requirement for each of these CD13 functions is tested in our various assays by 1) comparing wild-type and mutant enzymatically inactive CD13 used as agonists or to reconstitute CD13-depleted SF, as previously described for T cell chemotaxis assays (1); and by 2) treating cells that are the target of CD13 with pertussis toxin prior to use in various assays, to determine GPCR-dependence of the function under study.

The normalcy of the data is determined for each group, and then the Student's t test or ANOVA is used for statistical measurements. If the data show normal distribution, the results are analyzed using a Student's t-test. P values <0.05 are considered significant. If there is non-normal distribution of the data in animal models, a non-parametric data analysis approach is used. To compare differences in two groups (RA SFs with IgG and RA SFs with anti-human CD13 antibody), which are unpaired samples, a Mann-Whitney U test is used. This test is a non-parametric test for assessing whether two independent samples of observations come from the same distribution.

### Example 8

The Role of CD13 in MN Recruitment.

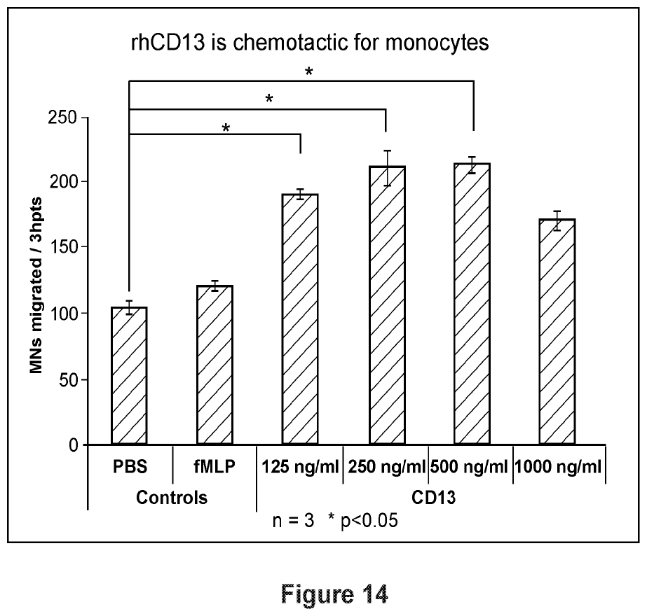

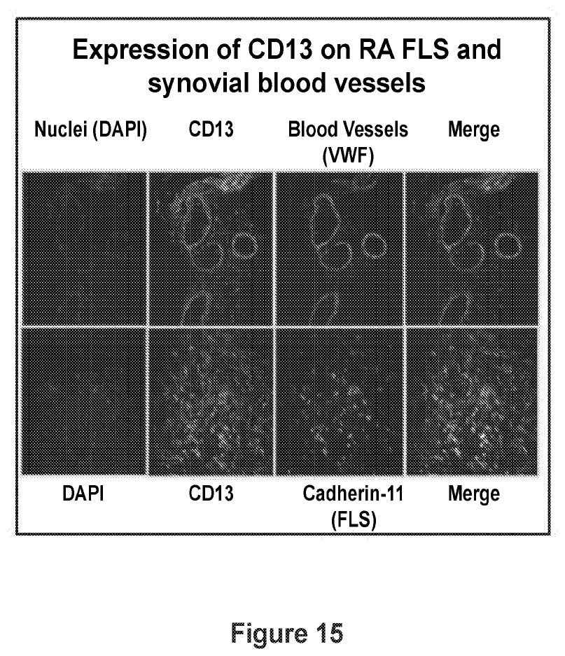

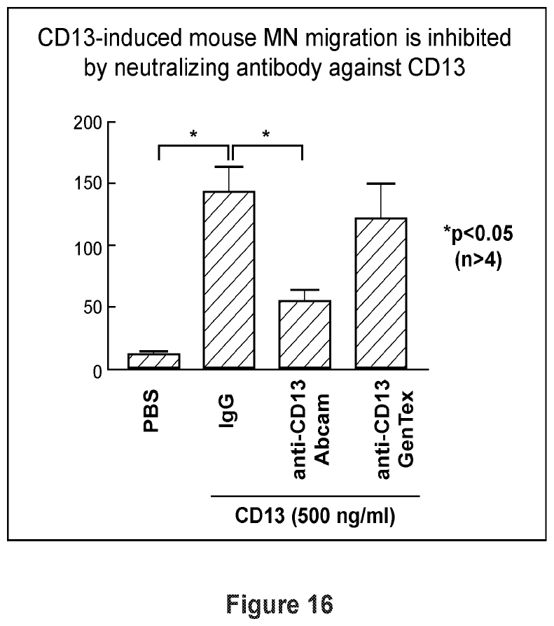

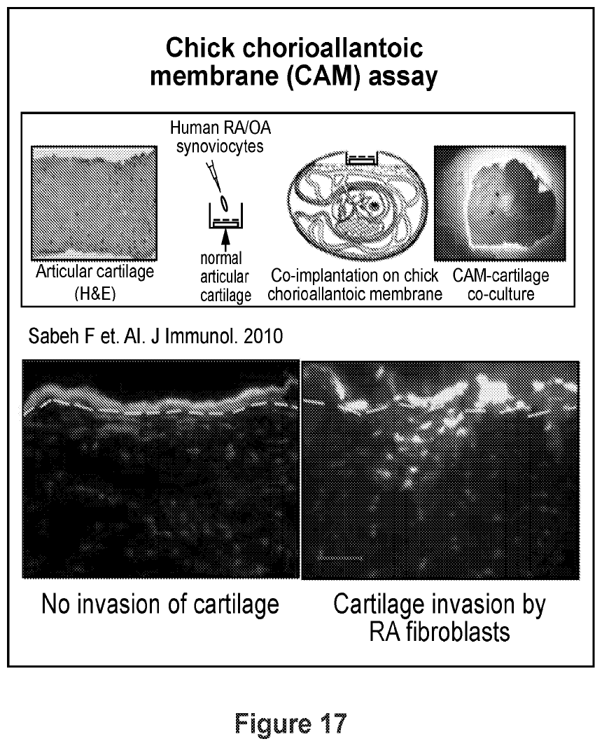

MN adhesion and recruitment into the sites of inflammation are critical steps in the pathogenesis of RA. MNs/macrophages are recruited to RA joints by a number of cytokines and chemokines (5, 11, 18, 19, 33, 66). Once these MNs/macrophages are recruited into the STs, they secrete proinflammatory and proangiogenic cytokines which results in the proliferation of ST membrane and more MN infiltration. In this way, a vicious cycle is formed which leads to persistence of inflammation in RA. The data disclosed herein show that recombinant human (rh)CD13 is a potent chemotactic factor for MNs in vitro (FIGS. 14, 16). The contribution of CD13 in MN recruitment in relation with RA is determined by performing a number of MN migration assays in vitro and in vivo.

Whether rhCD13 Induces MN Migration when Injected into Mouse Knee Joints

rhCD13 has been found to induce significantly higher MN migration in vitro compared to PBS, a negative control. To test the contribution of CD13 in MN recruitment in vivo, a mouse model of inflammatory arthritis will be used, with CD13 being directly injected into mouse knee joints (67).

To use the mouse model of inflammatory arthritis, female C57Bl/6 mice (8-10 weeks old, Harlan Laboratories) are anesthetized with ketamine (80-120 mg/kg body weight) and xylazine (5-10 mg/kg body weight) intraperitoneally, and the knee circumference is determined by caliper measurements before intra-articular injection, as described (67). The anesthetized mice receive 20 μl/knee joint of PBS and recombinant mouse CD13 (500 ng in 20 μl of PBS). Circumference measurements are obtained in a blinded manner. Mice are euthanized after 24 and 48 hours of the intraarticular injection. Mouse knees are measured before euthanasia. Hematoxylin and eosin (H&E) staining of the mouse knee cryosections is performed to determine the inflammatory response of CD13 (67). Immunofluorescence staining is also performed on cryosections using rat anti-mouse F4/80 antibody (GenTex) to detect MNs/macrophages. The number of F4/80-positive MNs/macrophages is calculated as the average number of cells in 3 fields (400X).

Whether RA SFs Depleted of CD13 Induce Less MN Recruitment in an RA ST-SCID Mouse Chimera

RA ST-SCID mouse chimera represents a unique way to study human tissue and human cells in vivo. This model is used to study normal human MN recruitment into RA STs engrafted in SCID mice in vivo in response to RA SFs depleted of CD13.

RA ST-SCID mouse chimera. RA ST is performed as described in Example 7 (68-72). After 4-6 weeks of engraftment, normal human PB MNs (2×10⁶ cells/100 μl of PBS) dye-tagged with PKH26 (Sigma Aldrich), are injected into each mouse via tail vein (68, 72). 95% pure MNs are usually obtained (68, 72). The viability of MNs is determined after labeling MNs. 5-6 RA SFs are pooled to minimize the variations present in CD13 expression in each fluid. Isotype IgG or anti-CD13 antibody (25 μg/ml) from Abcam is added to the mixture of SFs to deplete CD13. CD13-depleted or sham-depleted RA SFs (100 μl) are injected into each ST graft. TNF-α (200 ng/100 μl of PBS/graft) and PBS serve as positive and negative controls, respectively. The grafts are harvested at 48 hours post-injection and cryosections (6-8 μm thick) are examined using a fluorescence microscope. The number of migrated fluorescent MNs in the graft is assessed by counting the cells per three high power fields (hpfs). MNs migrated in response to sham-depleted RA SFs are compared with MNs migrated in response to CD13-depleted SFs as well as to MNs migrated in response to PBS.

Whether RA SFs Depleted of CD13 Induce Less MN Recruitment In Vitro

To evaluate the contribution of CD13 to recruitment of MNs in vitro, Normal human MN chemotaxis assays are performed in modified Boyden chambers using sham- or CD13-depleted RA SFs.

MN chemotaxis with RA SFs. MN chemotaxis assays are performed in a modified Boyden chamber, as described (58, 68, 73). Human RA SFs are incubated with anti-CD13 antibody (25 μg/ml, Abcam) or isotype control before performing chemotaxis (58, 68, 73). Each test group is assayed in quadruplicate. PBS and fMLP (200 nM) serve as negative and positive controls, respectively. MN chemotaxis assays are repeated at least 3-5 times with different donors, as previous experience has shown that this number is required to achieve statistical significance.

It is expected that there will be a more than 2-3 fold increase in mouse knee circumference injected with CD13 compared to PBS, consistent with the results found with other chemokines when injected into mouse knees (67). Increased mouse knee circumference in response to CD13 indicates more inflammation with increased MN ingress as well as increased production of proinflammatory mediators.

An RA ST-SCID model of inflammation is a good model to examine cellular homing, as this model has been used to investigate potential therapies directed against MN dependent diseases in vivo (68-71). The contribution of CD13 in MN recruitment in RA is unknown. We expect a more than 3-4 fold increase in MN ingress in STs engrafted in SCID mice in response to sham-depleted human RA SFs compared to CD13-depleted RA SFs or PBS (negative control). If this is the case, it indicates that CD13 is a key composition in MN migration in RA. MNs are dye-tagged with PKH26 and dye uptake is validated by fluorescence microscopy before being injected into SCID mice. Although no problems are expected with this dye, an alternate dye, CSFE (carboxyfluorescein diacetate, succinimidyl ester; Molecular Probe), is available if problems arise.

No major difficulties are apparent in isolating MNs from PB (68, 71, 74). A pure population of MNs is ensured by performing flow cytometry. If required, a negative MN selection kit from Miltenyi Biotech is available. As in Example 7, a mutant of CD13 that is enzymatically inactive is used to determine the role of the aminopeptidase function of CD13 in the actions of CD13 on MNs. MNs are also pretreated with pertussis toxin to determine whether, like T cells, they use a G-protein coupled receptor to respond to CD13.

Data will be analyzed as described in Example 7.

### Example 9

The Role of CD13 in MN Recruitment.

CD13 Involvement in Inflammation/Angiogenesis.

An experiment is performed to determine the role of CD13 in inflammation/angiogenesis in the K/B×N serum transfer arthritis model by treating mice with CD13 neutralizing antibody. RA is a prototype inflammatory disease characterized by leukocyte infiltration, which is in large part mediated by chemokines and cellular adhesion molecules. Angiogenesis is integral to the development of the inflamed RA ST pannus. Without angiogenesis, leukocyte recruitment could not occur. The experiment will take advantage of K/B×N serum transfer arthritis in which angiogenesis and MN ingress play a key role in arthritis development (75, 76). K/B×N serum transfer arthritis is caused by passive transfer of antibodies to glucose-6-phosphate isomerase and does not require participation of T and B cells (77). In contrast, macrophage-depleted mice are resistant to K/B×N serum transfer arthritis (78, 79). The K/B×N model is a robust one and has many advantages. For instance, the benefits of the K/B×N model compared to classic collagen-induced arthritis (CIA) include timeliness of arthritis development, consistency, and the severity and incidence of arthritis achieved. The K/B×N serum transfer model replicates many features of chronic RA in humans in a synchronized manner, such as synovial hypertrophy, infiltration of MNs/macrophages, pannus invasion, bone resorption, and joint ankyloses (75, 76, 80). In this model, CD13 is targeted using the antibody against mouse CD13 to assess the role of CD13 in the pathogenesis of RA. Anti-CD13 antibody significantly reduces migration of mouse MNs in response to CD13 in vitro (FIG. 16).

Induction K/B×N serum transfer arthritis. The K/B×N serum transfer model is used with female C57Bl/6 wild-type (wt) mice. K/B×N serum (150 μl) is injected intraperitoneally in wt mice (6- to 8-week-old, Harlan Laboratories) on day 0 and day two (76, 78, 79, 81-84). Anti-CD13 antibody (1 mg/mouse) is injected intraperitoneally (i/p) on day 0, 2, 4, 6, and 8 (85). Body weight, articular index scores, and ankle circumference are determined starting on day 0 and then every day.

Upon sacrifice on day 9, ankles and mouse blood are harvested and removed on ice. X-rays are also taken. Serum from each mouse is saved for quantifying proinflammatory/angiogenic factors. Some mouse ankles are skinned, weighed, and frozen in OCT at −80° C. to cut sections for IHC while others are homogenized to perform enzyme-linked immunosorbent assays (ELISAs) for cytokines and hemoglobin measurement. H&E-stained ankle sections are also scored by a pathologist blinded to experimental groups (86).

Whether there is a Decrease in Arthritis Severity and Joint Destruction in K/B×N Serum Transfer Arthritic Joints Treated with Anti-CD13 Antibody

Clinical Measurements.

By neutralizing the effects of CD13 in the K/B×N serum transfer arthritis model, it is determined whether there is decrease in clinical scores of mice treated with rat monoclonal CD13 antibody compared to isotype control treated mice. Clinical parameters are assessed on the days detailed above, and as described previously (61, 86-88).

The following scoring system is used: 0=Normal; 1=Mild redness and swelling of ankle, front paw, or individual digits; 2=Moderate redness and swelling of front paw or ankle; 3=Severe swelling of front paw or ankle; and 4=Maximally inflamed limb with involvement of multiple joints. This scoring system (85, 88-90) has been successfully used until day 9 (day of maximum arthritis).

Radiographic Scoring for Joint Inflammation and Destruction.

Ankles are promptly transferred to ice, and x-rays are taken and scored by a blinded observer, as described previously (61, 87, 88). Radiographs are scored for degree of bone erosion (0-4 scale) and joint space abnormality (0-3 scale), as has been done previously (61, 85, 88-90).

Histologic Analysis of Tissue Sections for Inflammation, Angiogenesis, Bone and Joint Erosions.

After euthanasia, ankles are embedded in OCT (Miles, Elkhart, Ind.), and sections (5 μm) are cut using a knife suitable for bone cutting and stained with hematoxylin and eosin. The synovial infiltrate, including MN/macrophages, polymorphonuclear cells, angiogenesis, and bone and joint erosions is scored on a scale of 0 to 5 for inflammation, MN ingress, angiogenesis, pannus formation, and bone erosion.

Whether there is a Decrease in MN Recruitment and Angiogenesis in Mouse Joints Treated with or without Anti-CD13 Antibody Treatment by IHC.

Cryosections (5 μm) are stained with markers that stain MNs, such as F4/80. Different groups, i.e., treated with or without anti-CD13 antibody, are compared. These cryosections are also stained with vonWillebrand factor, an EC marker, for the presence or absence of angiogenesis. Immunofluorescence is also performed using CD31, a marker for new blood vessels, and the number of blood vessels present in mouse joints treated with CD13 antibody is determined.

Whether there is Decreased Production of Proinflammatory/Angiogenic Factors in Mouse Arthritic Joint Homogenates in the Presence or Absence of Anti-CD13 Treatment.

Ankle Homogenates and ELISAs.

Ankles are homogenized, as described previously (61, 88, 89). Cytokine levels in ankle homogenates and mouse serum are determined using commercially available ELISA kits, such as TNF-α, IL-1β, IL-6, MCP-1/CCL2, KC/CXCL1, MIP-1α, bFGF, and VEGF (R&D Systems), according to the manufacturer's procedure. Differences in cytokine levels in the treated and non-treated groups are compared. ELISAs for the above-mentioned factors are performed because the factors play important roles in angiogenesis and inflammation in RA.

We expect attenuated joint inflammation in mice treated with purified anti-CD13 antibody in the K/B×N serum transfer arthritis model. Data disclosed herein indicates that CD13 is highly expressed by RA FLS and is significantly higher in RA SFs compared to OA. CD13 is angiogenic in vitro and in vivo and induces MN migration.

Another approach to assessing the role of CD13 in joint inflammation is the use of siRNA against CD13 to treat K/B×N serum transfer arthritis in mice. One of the major concerns about the use of siRNAs in in vivo models is that they are destroyed by RNAase in the body when injected systemically or locally. Modified siRNAs are designed that are not destroyed by RNAase, as these siRNAs have been used by others in in vivo assays without any major difficulties, such as siRNAs modified with 2′ fluoro pyrimidines or stealth RNA (92).

Statistical analysis is performed as described in Example 7.

### REFERENCES (FOR EXAMPLES 7-9)

1. Morgan R, Endres J, Behbahani-Nejad N, Phillips K, Ruth J H, Friday S C, et al. Expression and function of aminopeptidase N/CD13 produced by fibroblast-like synoviocytes in rheumatoid arthritis: role of CD13 in chemotaxis of cytokine-activated T cells independent of enzymatic activity. Arthritis & rheumatology. 2015; 67(1):74-85.
2. Szekanecz Z, Koch A E. Macrophages and their products in rheumatoid arthritis. Curr Opin Rheumatol. 2007; 19(3):289-95.
3. Ross R. Atherosclerosis—an inflammatory disease. N Engl J Med. 1999; 340(2):115-26.
4. Feldmann M, Brennan F M, Maini R N. Role of cytokines in rheumatoid arthritis. Annu Rev Immunol. 1996; 14:397-440.
5. Strand V, Kimberly R, Isaacs J D. Biologic therapies in rheumatology: lessons learned, future directions. Nat Rev Drug Discov. 2007; 6(1):75-92.
6. Feldmann M, Maini R N. Anti-TNF alpha therapy of rheumatoid arthritis: what have we learned? Annu Rev Immunol. 2001; 19:163-96.
7. Matsushima K, Larsen C G, DuBois G C, Oppenheim J J. Purification and characterization of a novel monocyte chemotactic and activating factor produced by a human myelomonocytic cell line. J Exp Med. 1989; 169(4):1485-90.
8. Hosaka S, Akahoshi T, Wada C, Kondo H. Expression of the chemokine superfamily in rheumatoid arthritis. Clin Exp Immunol. 1994; 97(3):451-7.
9. Szekanecz Z, Koch A E, Kunkel S L, Strieter R M. Cytokines in rheumatoid arthritis. Potential targets for pharmacological intervention. Drugs Aging. 1998; 12(5):377-90.
10. Szekanecz Z, Strieter R M, Kunkel S L, Koch A E. Chemokines in rheumatoid arthritis. Springer Semin Immunopathol. 1998; 20(1-2):115-32.
11. Koch A E, Kunkel S L, Strieter R M. Cytokines in rheumatoid arthritis. J Investig Med. 1995; 43(1):28-38.
12. Szekanecz Z, Szegedi G, Koch A E. Angiogenesis in rheumatoid arthritis: pathogenic and clinical significance. J Investig Med. 1998; 46(2):27-41.
13. Paleolog E M. Angiogenesis: a critical process in the pathogenesis of R A—a role for VEGF? Br J Rheumatol. 1996; 35(10):917-9.
14. Koch A E. The role of angiogenesis in rheumatoid arthritis: recent developments. Ann Rheum Dis. 2000; 59 Suppl 1:i65-71.
15. Shu Q, Amin M A, Ruth J H, Campbell P L, Koch A E. Suppression of endothelial cell activity by inhibition of TNFalpha. Arthritis Res Ther. 2012; 14(2):R88.
16. Kim S J, Chen Z, Chamberlain N D, Volin M V, Swedler W, Volkov S, et al. Angiogenesis in rheumatoid arthritis is fostered directly by toll-like receptor 5 ligation and indirectly through interleukin-17 induction. Arthritis Rheum. 2013; 65(8):2024-36.
17. Semerano L, Clavel G, Assier E, Denys A, Boissier M C. Blood vessels, a potential therapeutic target in rheumatoid arthritis? Joint Bone Spine. 2010.
18. Allavena P, Bianchi G, Zhou D, van Damme J, Jilek P, Sozzani S, et al. Induction of natural killer cell migration by monocyte chemotactic protein-1, -2 and -3. Eur J Immunol. 1994; 24(12):3233-6.
19. Carr M W, Roth S J, Luther E, Rose S S, Springer T A. Monocyte chemoattractant protein 1 acts as a T-lymphocyte chemoattractant. Proc Natl Acad Sci USA. 1994; 91(9):3652-6.
20. Koch A E, Harlow L A, Haines G K, Amento E P, Unemori E N, Wong W L, et al. Vascular endothelial growth factor. A cytokine modulating endothelial function in rheumatoid arthritis. J Immunol. 1994; 152(8):4149-56.
21. Nagashima M, Yoshino S, Ishiwata T, Asano G. Role of vascular endothelial growth factor in angiogenesis of rheumatoid arthritis. J Rheumatol. 1995; 22(9):1624-30.
22. Pickens S R, Volin M V, Mandelin A M, 2nd, Kolls J K, Pope R M, Shahrara S. I L-17 contributes to angiogenesis in rheumatoid arthritis. J Immunol. 2010; 184(6):3233-41.
23. Shahrara S, Pickens S R, Mandelin A M, 2nd, Karpus W J, Huang Q, Kolls J K, et al. IL-17-mediated monocyte migration occurs partially through C C chemokine ligand 2/monocyte chemoattractant protein-1 induction. J Immunol. 2010; 184(8):4479-87.
24. Shahrara S, Pickens S R, Dorfleutner A, Pope R M. IL-17 induces monocyte migration in rheumatoid arthritis. J Immunol. 2009; 182(6):3884-91.
25. De Bandt M, Ben Mandi M H, Ollivier V, Grossin M, Dupuis M, Gaudry M, et al. Blockade of vascular endothelial growth factor receptor I (VEGF-RI), but not VEGF-RII, suppresses joint destruction in the K/B×N model of rheumatoid arthritis. J Immunol. 2003; 171(9):4853-9.
26. Grosios K, Wood J, Esser R, Raychaudhuri A, Dawson J. Angiogenesis inhibition by the novel VEGF receptor tyrosine kinase inhibitor, PTK787/ZK222584, causes significant anti-arthritic effects in models of rheumatoid arthritis. Inflamm Res. 2004; 53(4):133-42.
27. Clavel G, Valvason C, Yamaoka K, Lemeiter D, Laroche L, Boissier M C, et al. Relationship between angiogenesis and inflammation in experimental arthritis. Eur Cytokine Netw. 2006; 17(3):202-10.
28. Afuwape A O, Feldmann M, Paleolog E M. Adenoviral delivery of soluble VEGF receptor 1 (sFlt-1) abrogates disease activity in murine collagen-induced arthritis. Gene Ther. 2003; 10(23):1950-60.
29. Sone H, Kawakami Y, Sakauchi M, Nakamura Y, Takahashi A, Shimano H, et al. Neutralization of vascular endothelial growth factor prevents collagen-induced arthritis and ameliorates established disease in mice. Biochem Biophys Res Commun. 2001; 281(2):562-8.
30. Ivashkiv L B. Cytokine expression and cell activation in inflammatory arthritis. Adv Immunol. 1996; 63:337-76.
31. Fava R A, Olsen N J, Spencer-Green G, Yeo K T, Yeo T K, Berse B, et al. Vascular permeability factor/endothelial growth factor (VPF/VEGF): accumulation and expression in human synovial fluids and rheumatoid synovial tissue. J Exp Med. 1994; 180(1):341-6.
32. Paleolog E M, Young S, Stark A C, McCloskey R V, Feldmann M, Maini R N. Modulation of angiogenic vascular endothelial growth factor by tumor necrosis factor alpha and interleukin-1 in rheumatoid arthritis. Arthritis Rheum. 1998; 41(7):1258-65.
33. Feldmann M, Maini R N, Bondeson J, Taylor P, Foxwell B M, Brennan F M. Cytokine blockade in rheumatoid arthritis. Adv Exp Med Biol. 2001; 490:119-27.
34. Maini R N, Breedveld F C, Kalden J R, Smolen J S, Davis D, Macfarlane J D, et al. Therapeutic efficacy of multiple intravenous infusions of anti-tumor necrosis factor alpha monoclonal antibody combined with low-dose weekly methotrexate in rheumatoid arthritis. Arthritis Rheum. 1998; 41(9):1552-63.
35. Look A T, Ashmun R A, Shapiro L H, Peiper S C. Human myeloid plasma membrane glycoprotein CD13 (gp150) is identical to aminopeptidase N. J Clin Invest. 1989; 83(4):1299-307.
36. Luan Y, Xu W. The structure and main functions of aminopeptidase N. Current medicinal chemistry. 2007; 14(6):639-47.
37. Sjostrom H, Noren O, Danielsen E M, Skovbjerg H. Structure of microvillar enzymes in different phases of their life cycles. Ciba Foundation symposium. 1983; 95:50-72.
38. Kehlen A, Langner J, Riemann D. Transforming growth factor-beta increases the expression of aminopeptidase N/CD13 mRNA and protein in monocytes and monocytic cell lines. Adv Exp Med Biol. 2000; 477:49-56.
39. Petrovic N, Bhagwat S V, Ratzan W J, Ostrowski M C, Shapiro L H. CD13/APN transcription is induced by RAS/MAPK-mediated phosphorylation of Ets-2 in activated endothelial cells. J Biol Chem. 2003; 278(49):49358-68.
40. Mina-Osorio P. The moonlighting enzyme CD13: old and new functions to target. Trends Mol Med. 2008; 14(8):361-71.
41. Favaloro E J, Browning T, Facey D. CD13 (GP150; aminopeptidase-N): predominant functional activity in blood is localized to plasma and is not cell-surface associated. Experimental hematology. 1993; 21(13):1695-701.
42. van Hensbergen Y, Broxterman H J, Hanemaaijer R, Jorna A S, van Lent N A, Verheul H M, et al. Soluble aminopeptidase N/CD13 in malignant and nonmalignant effusions and intratumoral fluid. Clin Cancer Res. 2002; 8(12):3747-54.
43. Villasenor-Cardoso M I, Frausto-Del-Rio D A, Ortega E. Aminopeptidase N (CD13) is involved in phagocytic processes in human dendritic cells and macrophages. BioMed research international. 2013; 2013:562984.
44. Mina-Osorio P, Ortega E. Aminopeptidase N (CD13) functionally interacts with FcgammaRs in human monocytes. J Leukoc Biol. 2005; 77(6):1008-17.
45. Mina-Osorio P, Winnicka B, O'Conor C, Grant C L, Vogel L K, Rodriguez-Pinto D, et al. CD13 is a novel mediator of monocytic/endothelial cell adhesion. J Leukoc Biol. 2008; 84(2):448-59.
46. Ghosh M, Gerber C, Rahman M M, Vernier K M, Pereira F E, Subramani J, et al. Molecular mechanisms regulating CD13-mediated adhesion. Immunology. 2014; 142(4):636-47.
47. Santos A N, Langner J, Herrmann M, Riemann D. Aminopeptidase N/CD13 is directly linked to signal transduction pathways in monocytes. Cell Immunol. 2000; 201(1):22-32.
48. Pasqualini R, Koivunen E, Kain R, Landenranta J, Sakamoto M, Stryhn A, et al. Aminopeptidase N is a receptor for tumor-homing peptides and a target for inhibiting angiogenesis. Cancer Res. 2000; 60(3):722-7.
49. Riemann D, Kehlen A, Langner J. CD13—not just a marker in leukemia typing. Immunol Today. 1999; 20(2):83-8.
50. Grujic M, Zavasnik-Bergant T, Pejler G, Renko M. Actinonin induces apoptosis in U937 leukemia cells. Cancer letters. 2005; 223(2):211-8.
51. Sekine K, Fujii H, Abe F. Induction of apoptosis by bestatin (ubenimex) in human leukemic cell lines. Leukemia. 1999; 13(5):729-34.
52. Scott D L, Wolfe F, Huizinga T W. Rheumatoid arthritis. Lancet. 2010; 376(9746):1094-108.
53. Carbonell J, Cobo T, Balsa A, Descalzo M A, Carmona L. The incidence of rheumatoid arthritis in Spain: results from a nationwide primary care registry. Rheumatology (Oxford). 2008; 47(7): 1088-92.
54. Pedersen J K, Kjaer N K, Svendsen A J, Horslev-Petersen K. Incidence of rheumatoid arthritis from 1995 to 2001: impact of ascertainment from multiple sources. Rheumatol Int. 2009; 29(4):411-5.
55. Folkman J. Angiogenesis in cancer, vascular, rheumatoid and other disease. Nat Med. 1995; 1(1):27-31.
56. Amin M A, Volpert O V, Woods J M, Kumar P, Harlow L A, Koch A E. Migration inhibitory factor mediates angiogenesis via mitogen-activated protein kinase and phosphatidylinositol kinase. Circ Res. 2003; 93(4):321-9.
57. Tsou P S, Ruth J H, Campbell P L, Isozaki T, Lee S, Marotte H, et al. A novel role for inducible Fut2 in angiogenesis. Angiogenesis. 2013; 16(1):195-205.
58. Volin M V, Woods J M, Amin M A, Connors M A, Harlow L A, Koch A E. Fractalkine: a novel angiogenic chemokine in rheumatoid arthritis. Am J Pathol. 2001; 159(4):1521-30.
59. Naber H P, Wiercinska E, Ten Dijke P, van Laar T. Spheroid assay to measure TGF-beta-induced invasion. Journal of visualized experiments: JoVE. 2011(57).
60. Dakouane-Giudicelli M, Brouillet S, Traboulsi W, Torre A, Vallat G, Si Nacer S, et al. Inhibition of human placental endothelial cell proliferation and angiogenesis by netrin-4. Placenta. 2015; 36(11): 1260-5 .
61. Amin M A, Campbell P L, Ruth J H, Isozaki T, Rabquer B J, Alex Stinson W, et al. A key role for Fut1-regulated angiogenesis and ICAM-1 expression in K/B×N arthritis. Ann Rheum Dis. 2015; 74(7):1459-66.
62. Park C C, Morel J C, Amin M A, Connors M A, Harlow L A, Koch A E. Evidence of IL-18 as a novel angiogenic mediator. J Immunol. 2001; 167(3):1644-53.
63. Amin M A, Rabquer B J, Mansfield P J, Ruth J H, Marotte H, Haas C S, et al. Interleukin 18 induces angiogenesis in vitro and in vivo via Src and Jnk kinases. Ann Rheum Dis. 2010; 69(12):2204-12.
64. Sabeh F, Fox D, Weiss S J. Membrane-type I matrix metalloproteinase-dependent regulation of rheumatoid arthritis synoviocyte function. J Immunol. 2010; 184(11):6396-406.
65. Amin M A, Mansfield P J, Pakozdi A, Campbell P L, Ahmed S, Martinez R J, et al. Interleukin-18 induces angiogenic factors in rheumatoid arthritis synovial tissue fibroblasts via distinct signaling pathways. Arthritis Rheum. 2007; 56(6):1787-97.
66. Koch A E, Kunkel S L, Harlow L A, Mazarakis D D, Haines G K, Burdick M D, et al. Macrophage inflammatory protein-1 alpha. A novel chemotactic cytokine for macrophages in rheumatoid arthritis. J Clin Invest. 1994; 93(3):921-8.
67. Yoshida K, Korchynskyi O, Tak P P, Isozaki T, Ruth J H, Campbell P L, et al. Citrullination of epithelial neutrophil-activating peptide 78/CXCLS results in conversion from a non-monocyte-recruiting chemokine to a monocyte-recruiting chemokine. Arthritis & rheumatology. 2014; 66(10):2716-27.
68. Amin M A, Ruth J H, Haas C S, Pakozdi A, Mansfield P J, Haghshenas J, et al. H-2g, a glucose analog of blood group H antigen, mediates mononuclear cell recruitment via Src and phosphatidylinositol 3-kinase pathways. Arthritis Rheum. 2008; 58(3):689-95.
69. Blades M C, Ingegnoli F, Wheller S K, Manzo A, Wahid S, Panayi G S, et al. Stromal cell-derived factor 1 (CXCL12) induces monocyte migration into human synovium transplanted onto SCID Mice. Arthritis Rheum. 2002; 46(3):824-36.
70. Matsuno H, Yudoh K, Uzuki M, Nakazawa F, Sawai T, Yamaguchi N, et al. Treatment with the angiogenesis inhibitor endostatin: a novel therapy in rheumatoid arthritis. J Rheumatol. 2002; 29(5):890-5.
71. Ruth J H, Haas C S, Park C C, Amin M A, Martinez R J, Haines G K, 3rd, et al. CXCL16-mediated cell recruitment to rheumatoid arthritis synovial tissue and murine lymph nodes is dependent upon the MAPK pathway. Arthritis Rheum. 2006; 54(3):765-78.
72. Ruth J H, Arendt M D, Amin M A, Ahmed S, Marotte H, Rabquer B J, et al. Expression and function of CXCL16 in a novel model of gout. Arthritis Rheum. 2010; 62(8):2536-44.
73. Kumar P, Hosaka S, Koch A E. Soluble E-selectin induces monocyte chemotaxis through Src family tyrosine kinases. J Biol Chem. 2001; 276(24):21039-45.
74. Amin M A, Haas C S, Zhu K, Mansfield P J, Kim M J, Lackowski N P, et al. Migration inhibitory factor up-regulates vascular cell adhesion molecule-1 and intercellular adhesion molecule-1 via Src, PI3 kinase, and NFkappaB. Blood. 2006; 107(6):2252-61.
75. Solomon S, Rajasekaran N, Jeisy-Walder E, Snapper S B, Illges H. A crucial role for macrophages in the pathology of K/B×N serum-induced arthritis. Eur J Immunol. 2005; 35(10):3064-73.
76. Scatizzi J C, Hutcheson J, Bickel E, Woods J M, Klosowska K, Moore T L, et al. p21Cip1 is required for the development of monocytes and their response to serum transfer-induced arthritis. Am J Pathol. 2006; 168(5):1531-41.
77. Korganow A S, Ji H, Mangialaio S, Duchatelle V, Pelanda R, Martin T, et al. From systemic T cell self-reactivity to organ-specific autoimmune disease via immunoglobulins. Immunity. 1999; 10(4):451-61.
78. Paniagua R T, Chang A, Mariano M M, Stein E A, Wang Q, Lindstrom T M, et al. c-Fms-mediated differentiation and priming of monocyte lineage cells play a central role in autoimmune arthritis. Arthritis Res Ther. 2010; 12(1):R32.
79. Mancardi D A, Jonsson F, lannascoli B, Khun H, Van Rooijen N, Huerre M, et al. The murine high-affinity IgG receptor FcgammaRlV is sufficient for autoantibody-induced arthritis. J Immunol. 2011; 186(4):1899-903.
80. LaBranche T P, Hickman-Brecks C L, Meyer D M, Storer C E, Jesson M I, Shevlin K M, et al. Characterization of the KRN cell transfer model of rheumatoid arthritis (KRN-CTM), a chronic yet synchronized version of the K/B×N mouse. Am J Pathol. 2010; 177(3):1388-96.
81. Watts G M, Beurskens F J, Martin-Padura I, Ballantyne C M, Klickstein L B, Brenner M B, et al. Manifestations of inflammatory arthritis are critically dependent on LFA-1. J Immunol. 2005; 174(6):3668-75.
82. Lee D M, Friend D S, Gurish M F, Benoist C, Mathis D, Brenner M B. Mast cells: a cellular link between autoantibodies and inflammatory arthritis. Science. 2002; 297(5587):1689-92.
83. Matsumoto I, Maccioni M, Lee D M, Maurice M, Simmons B, Brenner M, et al. How antibodies to a ubiquitous cytoplasmic enzyme may provoke joint-specific autoimmune disease. Nat Immunol. 2002; 3(4):360-5.
84. Wu H J, Sawaya H, Binstadt B, Brickelmaier M, Blasius A, Gorelik L, et al. Inflammatory arthritis can be reined in by CpG-induced DC-NK cell cross talk. J Exp Med. 2007; 204(8): 1911-22.
85. Shahrara S, Amin M A, Woods J M, Haines G K, Koch A E. Chemokine receptor expression and in vivo signaling pathways in the joints of rats with adjuvant-induced arthritis. Arthritis Rheum. 2003; 48(12):3568-83.
86. Isozaki T, Amin M A, Arbab A S, Koch A E, Ha C M, Edhayan G, et al. Inhibitor of DNA binding 1 as a secreted angiogenic transcription factor in rheumatoid arthritis. Arthritis Res Ther. 2014; 16(2):R68.
87. Koch A E, Volin M V, Woods J M, Kunkel S L, Connors M A, Harlow L A, et al. Regulation of angiogenesis by the C—X-C chemokines interleukin-8 and epithelial neutrophil activating peptide 78 in the rheumatoid joint. Arthritis Rheum. 2001; 44(1):31-40.
88. Woods J M, Amin M A, Katschke K J, Jr., Volin M V, Ruth J H, Connors M A, et al. Interleukin-13 gene therapy reduces inflammation, vascularization, and bony destruction in rat adjuvant-induced arthritis. Hum Gene Ther. 2002; 13(3):381-93.
89. Woods J M, Katschke K J, Volin M V, Ruth J H, Woodruff D C, Amin M A, et al. IL-4 adenoviral gene therapy reduces inflammation, proinflammatory cytokines, vascularization, and bony destruction in rat adjuvant-induced arthritis. J Immunol. 2001; 166(2):1214-22.
90. Ruth J H, Amin M A, Woods J M, He X, Samuel S, Yi N, et al. Accelerated development of arthritis in mice lacking endothelial selectins. Arthritis Res Ther. 2005; 7(5):R959-70.
91. Rahman M M, Ghosh M, Subramani J, Fong G H, Carlson M E, Shapiro L H. CD13 regulates anchorage and differentiation of the skeletal muscle satellite stem cell population in ischemic injury. Stem cells. 2014; 32(6):1564-77.
92. Filleur S, Courtin A, Ait-Si-Ali S, Guglielmi J, Merle C, Harel-Bellan A, et al. SiRNA-mediated inhibition of vascular endothelial growth factor severely limits tumor resistance to antiangiogenic thrombospondin-1 and slows tumor vascularization and growth. Cancer Res. 2003; 63(14):3919-22.

Each of the references listed above and cited throughout the disclosure is incorporated by reference herein in its entirety, or in relevant part, as would be apparent from context.

The disclosed subject matter has been described with reference to various specific embodiments and techniques. It should be understood, however, that many variations and modifications may be made while remaining within the spirit and scope of the disclosed subject matter.

What is claimed is:

1. A method of generating a binding partner specifically recognizing a cell-surface protein of a synovial fibroblast comprising:
(a) contacting at least one synovial fibroblast with Interleukin 17 to stimulate the at least one synovial fibroblast;
(b) administering an immunogenic amount of the at least one synovial fibroblast to an immunocompetent host organism; and
(c) obtaining an antibody specifically recognizing a cell-surface protein of the synovial fibroblast.

2. The method of claim 1 wherein the binding partner is a monoclonal antibody or binding fragment thereof.

3. The method of claim 2 wherein the antibody is antibody 1D7, or a binding fragment thereof.

4. The method of claim 1 wherein the cell-surface protein is localized on an episome.

5. The method of claim 4 wherein the episome is 30-130 nm in diameter.

6. The method of claim 1 wherein the cell-surface protein is a human protein.

7. The method of claim 1 wherein the cell-surface protein is CD13.

8. A method of measuring the concentration of CD13 in a sample comprising
(a) contacting the sample with an anti-CD13 antibody, or binding fragment thereof, produced by the method of claim 7; and
(b) measuring the concentration of CD13 in the sample based on the extent of binding of anti-CD13 antibody, or binding fragment thereof.

9. The method of claim 7 that is an ELISA assay.

10. A method of treating an autoimmune disorder comprising administering an effective amount of an inhibitor of CD13.

11. The method of claim 10 wherein the inhibitor is an anti-CD13 antibody or binding fragment thereof.

12. The method of claim 11 wherein the anti-CD13 antibody is antibody 1D7 or a binding fragment thereof.

13. A method of treating an autoimmune disorder in a subject comprising administering an effective amount of an inhibitor of CD13 cleavage from a cell membrane.

14. The method of claim 13 wherein the autoimmune disorder is rheumatoid arthritis.

15. The method of claim 13 wherein the cell membrane is an exosome membrane.

16. The method of claim 13 wherein the inhibitor reduces the protein cleavage activity of a matrix metalloproteinase.

17. The method of claim 16 wherein the matrix metalloproteinase is selected from the group consisting of MMP14, MMP15, MMP16, MMP17, ADAM10, ADAM15 and ADAM17.

18. The method of claim 17 wherein the matrix metalloproteinase is MMP14.

19. The method of claim 16 wherein the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.

20. A method of inhibiting the migration of a cytokine-activated cell in a subject comprising administering an effective amount of a CD13 inhibitor.

21. The method of claim 20 wherein the cell is an endothelial cell, a monocyte or a T-cell.

22. The method of claim 20 wherein the inhibitor is an anti-CD13 antibody or binding fragment thereof.

23. The method of claim 22 wherein the anti-CD13 antibody is antibody 1D7 or a binding fragment thereof.

24. The method of claim 20 wherein the CD13 inhibitor is an inhibitor of a matrix metalloproteinase.

25. The method of claim 24 wherein the matrix metalloproteinase is MMP-14.

26. The method of claim 24 wherein the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.

27. A method of inhibiting angiogenesis in a subject comprising administering an effective amount of a CD13 inhibitor.

28. The method of claim 27 wherein the inhibitor is an anti-CD13 antibody or binding fragment thereof.

29. The method of claim 28 wherein the anti-CD13 antibody is antibody 1D7 or a binding fragment thereof.

30. The method of claim 27 wherein the CD13 inhibitor is an inhibitor of a matrix metalloproteinase.

31. The method of claim 30 wherein the matrix metalloproteinase is MMP-14.

32. The method of claim 30 wherein the inhibitor is selected from the group consisting of tissue inhibitor of metalloproteinase 1 (TIMP-1), tissue inhibitor of metalloproteinase 2 (TIMP-2), tissue inhibitor of metalloproteinase 3 (TIMP-3), GM6001, batimastat, llomastat, marimastat, periostat, a2-macroglobulin, catechin, gold salts, MMI-270, MMI-166, ABT-770, prinomastat, RS-130830, 239796-97-5, rebimastat, tanomastat, Ro 28-2653, 556052-30-3, 848773-43-3, 420121-84-2, 544678-85, 868368-30-3, doxycycline and COL-3.
